(19) United States
(12) Patent Application Publication
(10) Pub. No.: US 2025/0354996 A1
(43) Pub. Date: Nov. 20, 2025

(54) SIGNAL AMPLIFICATION AND MULTIPLEXING USING MASS TAGS FOR IA-LC-MS/ MS BASED ASSAYS

(71) Applicant: Genentech, Inc., South San Francisco, CA (US)

(72) Inventors: Helen Suk-Yin DAVIS, San Mateo, CA (US); Sukjoon HYUNG, Burlingame, CA (US); Surinder KAUR, Lafayette, CA (US); Ola M. SAAD, Walnut Creek, CA (US)

(73) Assignee: Genentech, Inc., South San Francisco, CA (US)

(21) Appl. No.: 19/207,921

(22) Filed: May 14, 2025

Related U.S. Application Data
(63) Continuation of application No. PCT/US2023/080388, filed on Nov. 17, 2023.
(60) Provisional application No. 63/426,672, filed on Nov. 18, 2022.

(51) Int. Cl. G01N 33/68; C12Q 1/37; G01N 1/34; G01N 33/543

(52) U.S. Cl. CPC G01N 33/6848; C12Q 1/37; G01N 1/34; G01N 33/54313; G01N 2333/95

(57) ABSTRACT

Provided herein are detection reagents and assays for detecting and/or quantitating an analyte in a sample by liquid chromatography/mass spectrometry (LS/MS). In some embodiments, the detection reagent is a compound having the formula wherein: B is a base detection moiety that binds the analyte, L is a cleavable linker, T is a tag suitable for mass spectroscopy, n is an integer greater than or equal to 1, and p is an integer greater than or equal to 1.

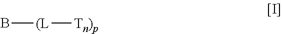

### CROSS-REFERENCE TO RELATED APPLICATION

This application is a continuation of International Application No. PCT/US2023/080388, filed internationally on Nov. 17 2023, which claims priority to U.S. Provisional Application No. 63/426,672, filed Nov. 18, 2022, entitled “SIGNAL AMPLIFICATION AND MULTIPLEXING USING MASS TAGS FOR IA-LC-MS/MS BASED ASSAYS,” the contents of which are incorporated by reference in their entirety.

### REFERENCE TO AN ELECTRONIC SEQUENCE LISTING

The contents of the electronic sequence listing (146392057601SEQLIST.xml; Size: 2,326 bytes; and Date of Creation: May 8, 2025) is herein incorporated by reference in its entirety.

### FIELD

The present disclosure in some aspects relates to methods of detecting one or more analytes such as polypeptide analytes in a sample. The present disclosure also relates to detection reagents used in the methods disclosed herein.

### BACKGROUND

There is an increasing demand for sensitive methods for detecting biotherapeutics in the background of complicated biological matrices.

Current immunoassay methods are often limited by the availability of specific high quality custom reagents, including the time required to generate these as well as their quality and lot-to-lot variability. For example, ligand binding assays (LBAs) generally use one or more carefully selected monoclonal or polyclonal antibody reagents to achieve the sensitivity and selectivity needed for the analyte of interest. Reagents may take 3-6 months to generate for one polypeptide analyte. Current IA-LC-MS/MS methods measure analytes directly without a signal amplification step, which results in the lack of gain in signal/noise ratio. For example, a protein analyte is first contacted with a protease that results in the generation of a heterogeneous population of peptide fragments that are used as surrogate peptides for mass spectrometry (MS) detection. There are needs for improved assays and reagents to achieve sensitivity down to ˜pg/mL levels and also the ability to take advantage of relatively well characterized reagents for biomarker assays, which can be further multiplexed without losing assay sensitivity. The present disclosure addresses these and other needs.

### BRIEF SUMMARY

In some embodiments, provided herein are methods, compositions (e.g., detection reagents), and kits for IA-LC-MS/MS (Immunoaffinity Liquid Chromatography with tandem Mass Spectrometry). In some embodiments, the assays described herein combine the signal amplification of a ligand binding assay and the robustness of LC-MS/MS to achieve sensitivity at the ˜pg/mL level. In some embodiments, the assays described herein enable the use of relatively well characterized reagents in a multiplexed assay format without losing assay sensitivity. In some embodiments, the assays described herein comprise the steps of capturing a target polypeptide (e.g., a peptide or protein such as an antibody), e.g., to enrich the target polypeptide on a bead using immunoaffinity, and labeling the target polypeptide with a binding reagent comprising cleavable linkers each linking one or more molecules of one or more tags to a base detection moiety that binds to the target polypeptide. Since each binding reagent can comprise multiple copies of a tag corresponding to one molecule of the target polypeptide, by detecting molecules of the tag instead of the target polypeptide itself or peptide fragments thereof, MS signals can be amplified. In addition, by analyzing multiple copies of the same molecule (e.g., the tag) instead of a heterogeneous population of digested peptide fragments, higher signal-to-noise (S/N) ratios in the MS detection can be achieved. By quantification of a unique tag that is specifically associated with the target polypeptide (e.g., by labeling each target polypeptide with multiple copies of one particular tag) and that provides a high S/N ratio, the MS assay can be configured to detect multiple target polypeptides by using multiple, polypeptide-specific tags for multiplexing, where MS signals corresponding to different target polypeptide can be resolved from each other (see e.g., FIG. 1 (middle panel) and FIG. 2 (bottom panel)) showing peaks for Tags 1-4 corresponding to peptide analytes 1-4, respectively).

In one aspect, provided herein is a method for detecting an analyte (A) in a sample, the method comprising a) affinity enrichment by contacting the sample with a capture reagent to generate a captured analyte complex, wherein the capture reagent comprises a binding reagent that binds the analyte, b) contacting the captured analyte complex with a detection reagent, wherein the detection reagent binds the captured analyte complex, wherein the detection reagent is a compound having the formula B-(L-Tₙ)ₚ wherein: B is a base detection moiety that binds the analyte, L is a cleavable linker, T is a tag suitable for mass spectroscopy, n is an integer greater than or equal to 1, and p is an integer greater than or equal to 1; c) washing the captured analyte complex with a buffer to remove unbound detection reagent; d) subjecting the sample from step c) under conditions to cleave the linker to release the tag, and e) analyzing the sample from step d) for presence of the released tag by mass spectroscopy.

In one aspect, provided herein is a method for quantifying an analyte (A) in a sample, the method comprising a) affinity enrichment by contacting the sample with a capture reagent to generate a captured analyte complex, wherein the capture reagent comprises a binding reagent that binds the analyte, b) contacting the captured analyte complex with a detection reagent, wherein the detection reagent binds the captured analyte complex, wherein the detection reagent is a compound having the formula B-(L-Tₙ)ₚ wherein: B is a base detection moiety that binds the analyte, L is a cleavable linker, T is a tag suitable for mass spectroscopy, n is an integer greater than or equal to 1, and p is an integer greater than or equal to 1; c) washing the captured analyte complex with a buffer to remove unbound detection reagent; d) subjecting the sample from step c) under conditions to cleave the linker to release the tag, and e) quantifying the released tag by mass spectroscopy.

In some embodiments, B does not bind the capture reagent. In some embodiments, the plurality of capture reagents is one or more of an antigen binding protein, an antibody, a soluble receptor, a protein A, a protein G, a protein L, or an extracellular domain.

In some embodiments, one or more of the capture agents is immobilized on a solid support. In some embodiments, the solid support is a bead.

In some embodiments, the base detection moiety is an antigen binding protein, an antibody, or a soluble receptor.

In some embodiments, n is 6-18. In some embodiments, p is 1-8. In some embodiments, p is 6-18. In some embodiments, n is 1-8. In some embodiments, p is 1-8 and n is 6-18. In some embodiments, p is 2 and n is 9.

In some embodiments, the linker is cleavable by an enzyme or by a chemical. In some embodiments, the base detection moiety is not cleaved under the conditions to cleave the linker. In some embodiments, the enzyme is a protease. In some embodiments, the protease is an endopeptidase. In some embodiments, the endopeptidase is papain.

In some embodiments, the base detection moiety does not comprise an amino acid sequence that undergoes chemical cleavage or protease cleavage.

In some embodiments, the tag is one or more of: a) a molecule that can be ionized and vaporized for analysis by mass spectrometry, b) a molecule with a controlled conjugation site, c) unique from other peptides in the sample, d) provides a good LC-MS response, e) does not compromise binding to the analyte when bound to base, f) has the capacity to functionalize for a multiplexed assay; and g) monitorable by a precursor ion-product ion pair ranging from 0-2000 m/z→0-2000 m/z.

In some embodiments, the detection reagent binds the analyte at a different site than the capture polypeptide.

In one aspect, provided herein is a method for detecting an analyte in a sample, the method comprising a) contacting the sample with a capture reagent to generate a captured analyte complex, wherein the capture reagent comprises a binding reagent that binds the analyte, and wherein the capture antibody is immobilized on a solid support, b) contacting the captured analyte complex with a detection reagent, wherein the detection reagent binds the captured analyte complex, wherein the detection reagent is a compound having the formula B-(L-Tₙ)ₚ wherein: B is a base detection moiety that binds the analyte, L is a cleavable linker, T is a tag suitable for mass spectroscopy, n is an integer greater than or equal to 1, and p is an integer greater than or equal to 1; c) washing the captured analyte complex with a buffer to remove unbound detection reagent; d) subjecting the sample from step c) to papain to cleave the linker to release the tag from the detection antibody, e) analyzing the sample from step d) for presence of the released tag by mass spectroscopy.

In one aspect, provided herein is a method for quantifying an analyte in a sample, the method comprising a) contacting the sample with a capture reagent to generate a captured analyte complex, wherein the capture reagent comprises a binding reagent that binds the analyte, and wherein the capture antibody is immobilized on a solid support, b) contacting the captured analyte complex with a detection reagent, wherein the detection reagent binds the captured analyte complex, wherein the detection reagent is a compound having the formula B-(L-Tₙ)ₚ wherein: B is a base detection moiety that binds the analyte, L is a cleavable linker, T is a tag suitable for mass spectroscopy, n is an integer greater than or equal to 1, and p is an integer greater than or equal to 1; c) washing the captured analyte complex with a buffer to remove unbound detection reagent; d) subjecting the sample from step c) to papain to cleave the linker to release the tag from the detection antibody, e) quantifying the released tag by mass spectroscopy.

In some embodiments, the analyte is a therapeutic polypeptide, a therapeutic antibody, or a biomarker.

In one aspect, provided herein is a method for detecting a plurality of analytes in a sample, the method comprising a) contacting the sample with a plurality of capture reagents to generate a plurality of captured analyte complexes, wherein the plurality of capture reagents comprises a plurality of capture polypeptides that bind the plurality of analytes, b) contacting the plurality of captured analyte complexes with a plurality of detection reagents, wherein the plurality of detection reagents bind the plurality of captured analyte complexes, wherein each detection reagent in the plurality of detection reagents is a compound having the formula B-(L-Tₙ)ₚ wherein: B is a base detection moiety that binds the analyte, L is a cleavable linker, T is a tag suitable for mass spectroscopy, n is an integer greater than or equal to 1, and p is an integer greater than or equal to 1, wherein each tag in the plurality of detection reagents can be distinguished from one another by mass spectroscopy; c) washing the plurality of captured analyte complex with a buffer to remove unbound detection reagent; d) subjecting the sample from step c) under conditions to cleave the linker to release the tags from the plurality of detection reagents, e) analyzing the sample from step d) for presence of the plurality of released tags by mass spectroscopy.

In one aspect, provided herein is a method for quantifying a plurality of analytes in a sample, the method comprising a) contacting the sample with a plurality of capture reagents to generate a plurality of captured analyte complexes, wherein the plurality of capture reagents comprises a plurality of capture polypeptides that bind the plurality of analytes, b) contacting the plurality of captured analyte complexes with a plurality of detection reagents, wherein the plurality of detection reagents bind the plurality of captured analyte complexes, wherein each detection reagent in the plurality of detection reagents is a compound having the formula B-(L-Tₙ)ₚ wherein: B is a base detection moiety that binds the analyte, L is a cleavable linker, T is a tag suitable for mass spectroscopy, n is an integer greater than or equal to 1, and p is an integer greater than or equal to 1, wherein each tag in the plurality of detection reagents can be distinguished from one another by mass spectroscopy; c) washing the captured analyte complex with a buffer to remove unbound detection reagent; d) subjecting the sample from step c) under conditions to cleave the linker to release the tags from the plurality of detection reagents, e) quantifying the plurality of released tags by mass spectroscopy.

In some embodiments, B does not bind the capture reagent.

In some embodiments, the plurality of capture reagents is one or more of an antigen binding protein, an antibody, a soluble receptor, a protein A, a protein G, a protein L, or an extracellular domain.

In some embodiments, one or more of the capture agents is immobilized on a solid support. In some embodiments, the solid support is a bead.

In some embodiments, the base detection moiety is an antigen binding protein, an antibody, or a soluble receptor. In some embodiments, n is 6-18. In some embodiments, p is 1-8.In some embodiments, p is 6-18. In some embodiments, n is 1-8.

In some embodiments, the linkers of the plurality of detection reagents are cleavable by enzymes or by chemicals. In some embodiments, the plurality of base detection moieties are not cleaved under the conditions to cleave the linker. In some embodiments, the enzyme is a protease. In some embodiments, linkers of the plurality of detection reagents are cleavable by one or more proteases. In some embodiments, one or more of the proteases is an endopeptidase. In some embodiments, the one or more endopeptidases is papain.

In some embodiments, one or more of the base detection moieties of the plurality of base detection moieties do not comprise an amino acid sequence that undergoes chemical cleavage or protease cleavage. In some embodiments, one or more of the linkers in the plurality of detection reagents are cleaved by the same protease or chemical.

In some embodiments, all the linkers in the plurality of detection reagents are the same.

In some embodiments, the tag for each detection reagent of the plurality of detection reagents is one or more of a) a molecule that can be ionized and vaporized for analysis by mass spectrometry, b) a molecule with a controlled conjugation site, c) unique from other peptides in the sample, d) provides a good LC-MS response, e) does not compromise binding to the analyte when bound to base, or f) has the capacity to functionalize for a multiplexed assay.

In one aspect, provided herein is a method for detecting a plurality of analytes in a sample, the method comprising a) contacting the sample with a plurality of capture reagents to generate a plurality of captured analyte complexes, wherein the plurality of capture reagents comprises a plurality of capture antibodies that bind the plurality of analytes, and wherein the plurality of capture antibodies are immobilized on a solid support, b) contacting the plurality of captured analyte complexes with a plurality of detection reagents, wherein the plurality of detection reagents bind the plurality of captured analyte complexes, wherein each detection reagent in the plurality of detection reagents is a compound having the formula B-(L-Tₙ)ₚ wherein: B is a base detection moiety that binds the analyte, L is a cleavable linker, T is a tag suitable for mass spectroscopy, n is an integer between 1 and 50, and p is an integer between 1 and 50, wherein each tag in the plurality of detection reagents can be distinguished from one another by mass spectroscopy; c) washing the plurality of captured analyte complexes with a buffer to remove unbound detection reagents; d) subjecting the sample from step c) to papain to cleave the linker to release the tags from the plurality of detection reagents, and e) analyzing the sample from step d) for presence of the plurality of released tags by mass spectroscopy.

In one aspect, provided herein is a method for quantifying a plurality of analytes in a sample, the method comprising a) contacting the sample with a plurality of capture reagents to generate a plurality of captured analyte complexes, wherein the plurality of capture reagents comprises a plurality of capture antibodies that bind the plurality of analytes, and wherein the plurality of capture antibodies are immobilized on a solid support, b) contacting the plurality of captured analyte complexes with a plurality of detection reagents, wherein the plurality of detection reagents bind the plurality of captured analyte complexes, wherein each detection reagent in the plurality of detection reagents is a compound having the formula B-(L-Tₙ)ₚ wherein: B is a base detection moiety that binds the analyte, L is a cleavable linker, T is a tag suitable for mass spectroscopy, n is an integer between 1 and 6, and p is an integer between 1 and 8, wherein each tag in the plurality of detection reagents can be distinguished from one another by mass spectroscopy; c) washing the plurality of captured analyte complexes with a buffer to remove unbound detection reagents; d) subjecting the sample from step c) to papain to cleave the linker to release the tags from the plurality of detection reagents, and e) quantifying the plurality of released tags by mass spectroscopy.

In some embodiments, the plurality of analytes comprise two or more of therapeutic polypeptides, therapeutic antibodies, and/or biomarkers.

In one aspect, provided herein is a detection reagent for use in detecting an analyte in a sample by mass spectroscopy, wherein the detection reagent is a compound having the formula B-(L-Tₙ)ₚ wherein: B is a base detection moiety that binds the analyte, L is a cleavable linker, T is a tag suitable for mass spectroscopy, n is an integer greater than or equal to 1, and p is an integer greater than or equal to 1.

In some embodiments, the base detection moiety is an antigen binding protein, an antibody, or a soluble receptor.

In some embodiments, n is 6-18. In some embodiments, n is 1-8. In any one of the embodiments herein, n can be 1, 2, 3, 4, 5, 6, 7, 8, 9, 10, 11, 12, 13, 14, 15, 16, 17, or 18.

In some embodiments, p is 1-8. In some embodiments, p is 6-18. In any one of the embodiments herein, p can be 1, 2, 3, 4, 5, 6, 7, 8, 9, 10, 11, 12, 13, 14, 15, 16, 17, or 18.

In some embodiments, the linker is cleavable by an enzyme or by a chemical.

In some embodiments, the base detection moiety is not cleaved under the conditions to cleave the linker.

In some embodiments, the enzyme is a protease.

In some embodiments, the protease is an endopeptidase.

In some embodiments, the endopeptidase is papain.

In some embodiments, the base detection moiety does not comprise an amino acid sequence that undergoes chemical cleavage or protease cleavage.

In some embodiments, the tag is one or more of a) a molecule that can be ionized and vaporized for analysis by mass spectrometry, b) a molecule with a controlled conjugation site, c) unique from other peptides in the sample, d) provides a good LC-MS response, e) does not compromise binding to the analyte when bound to base, or f) has the capacity to functionalize for a multiplexed assay.

In some embodiments, the detection reagent binds the analyte at a different site than the capture polypeptide.

In one aspect, provided herein is a detection reagent for use in detecting an analyte in a sample by mass spectroscopy, wherein the detection reagent is a compound having the formula B-(L-Tₙ)ₚ wherein: B is a base detection moiety that binds the analyte, L is a cleavable linker, T is a tag suitable for mass spectroscopy, n is an integer greater than or equal to 1, and p is an integer greater than or equal to 1.

In some embodiments, the plurality of analytes are two or more of therapeutic polypeptides, therapeutic antibodies, and/or biomarkers.

In one aspect, provided herein is a composition comprising the detection reagent of any one of the detection reagents provided herein.

In one aspect, provided herein is a composition comprising a plurality of detection reagents of any one of the detection reagents provided herein, wherein each detection reagent in the plurality of detection reagents binds a different analyte and comprises a different tag.

In one aspect, provided herein is a kit for use in the method of any one of the methods provided herein.

In one aspect, provided herein is a kit comprising the detection reagent of any one of the detection reagents provided herein.

In one aspect, provided herein is a kit comprising any one of the compositions provided herein.

In one aspect, provided herein is a method for detecting an analyte in a sample, comprising: a) contacting the sample with a capture reagent to generate a captured analyte complex, wherein the capture reagent comprises a binding reagent that binds the analyte, b) contacting the captured analyte complex with a detection reagent, wherein the detection reagent binds the captured analyte complex, wherein the detection reagent comprises a base detection moiety that binds the analyte, wherein the base detection moiety is directly or indirectly linked to a plurality of tags, and wherein the plurality of tags are cleavable from the detection reagent; c) separating unbound detection reagent from the captured analyte complex bound to the detection reagent; d) cleaving the plurality of tags from the detection reagent bound to the captured analyte complex, thereby releasing the tags, and e) analyzing the released tags by mass spectroscopy.

In any of the preceding embodiments, analyzing the released tags by mass spectroscopy can comprise generating a signal-to-noise ratio (S/N) of the released tags.

In any of the preceding embodiments, the S/N of the released tags detected by mass spectroscopy can be about 2, 3, 4, 5, 6, 7, 8, 9, 10, 11, 12, 13, 14, 15, 16, 17, 18, 19, 20, or more.

In any of the preceding embodiments, the lower limit of quantitation (LLOQ) for the analyte can be between about 0.1 and about 20 ng/mL, between about 0.2 and about 15 ng/ml, or between about 0.5 and about 10 ng/mL.

### BRIEF DESCRIPTION OF THE DRAWINGS

FIG. 1 shows a schematic representation of quantifying an analyte (e.g., a polypeptide analyte). The top panel shows the generic steps of the IA-LC-MS/MS (Immunoaffinity Liquid Chromatography with tandem Mass Spectrometry) method for detecting a polypeptide analyte using one or more surrogate peptides. The middle panel shows exemplary steps for the IA-LC-MS/MS method provided herein for detecting an analyte (e.g., a polypeptide analyte) using a detection reagent comprising a base detection moiety (e.g., a polypeptide), cleavable linker and tag. The bottom panel shows an ELISA assay format whereby a polypeptide analyte is immobilized on a solid support for binding to a detection antibody conjugated to HRP (horseradish peroxidase) for colorimetric detection.

FIG. 2 shows a schematic representation of multiplex assays for quantifying multiple analytes (e.g., polypeptide analytes). The top panel shows the generic steps for a multiplex immunoassay where a mixture of polypeptide analytes are detected individually by a detection antibody conjugated to a fluorophore. The bottom panel shows exemplary steps for the IA-LC-MS/MS method provided herein for quantifying a mixture of analytes (e.g., polypeptide analytes) using multiple detection reagents comprising a base detection moiety (e.g., a polypeptide), cleavable linker and tag.

FIG. 3 shows exemplary steps for the IA-LC-MS/MS method provided herein for capturing, detecting, and quantifying an analyte (e.g., a polypeptide analyte). Capture of a polypeptide analyte (e.g., an extracellular domain (ECD) of a receptor) begins with the introduction of a capture reagent, wherein the capture reagent comprises a binding agent (e.g., an antibody) that binds the analyte and is conjugated to streptavidin coated magnetic beads, followed by isolation of the polypeptide analyte (e.g., ECD) by magnetic separation. Next, the detection reagent conjugated to a variable number of tags is added, followed by cleaving molecules of the tag off the detection reagent, and analysis by LC-MS/MS using the tag (e.g., MMAE) for quantification of the polypeptide analyte (e.g., ECD).

FIG. 4 shows exemplary steps for the IA-LC-MS/MS method provided herein for capturing an analyte (e.g., an antibody) whereby a capture reagent on streptavidin coated magnetic beads is introduced to the analyte. A detection reagent conjugated to a variable number of tags (e.g., Leucine enkephalin: YGGFL; SEQ ID NO:1); e.g., n=9 and p=2 in formula B-(L-Tₙ)ₚ for the detection reagent) is then introduced to the analyte, followed by magnetic separation, cleavage of the tag molecules, and analysis by IA-LC-MS/MS using the tag for quantification of the analyte.

FIG. 5 shows the linearity of detection, plotting the peak area counts of tag (e.g., MMAE) versus the concentration of an analyte in phosphate buffered saline. Increasing the numbers of tag molecules in the detection reagent results in higher analyte peak area counts.

FIGS. 6A-6F show a comparison of extracted ion chromatograms using the current IA-LC-MS/MS method (FIG. 6A) and the exemplary steps for the IA-LC-MS/MS method provided herein (FIGS. 6B-6F). FIG. 6A shows the LLOQ level of a polypeptide analyte (e.g., an extracellular domain-ECD). (FIGS. 6B-6F) show the LLOQ level of a tag (e.g., MMAE) using detection reagent having different tag-to-base detection moiety ratios (e.g., drug-to-antibody ratios, DARs). There is higher signal/noise (S/N) ratio for MS detection of the tag compared to MS detection of the polypeptide analyte (ECD P-1).

### DETAILED DESCRIPTION

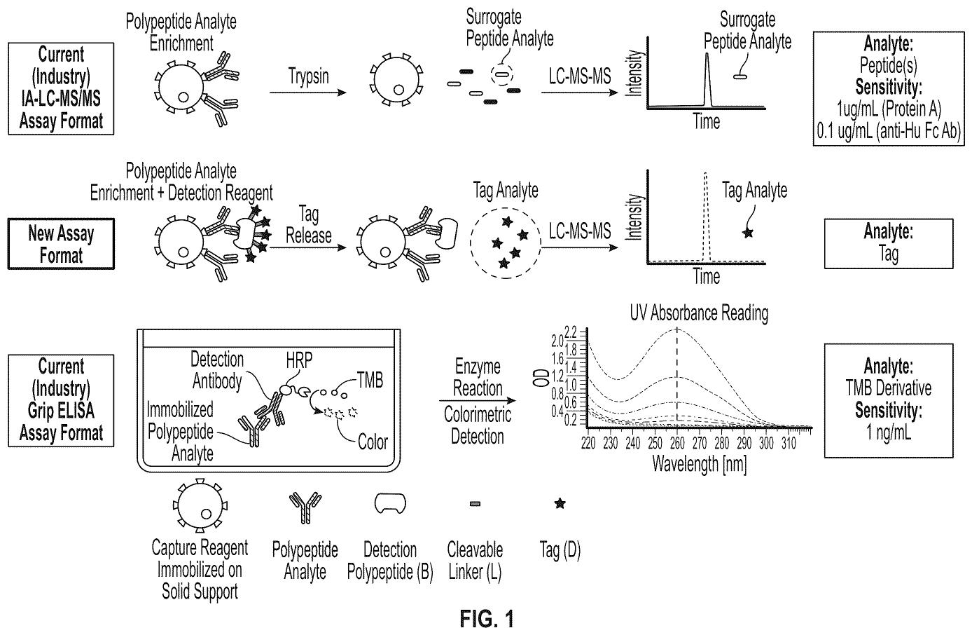

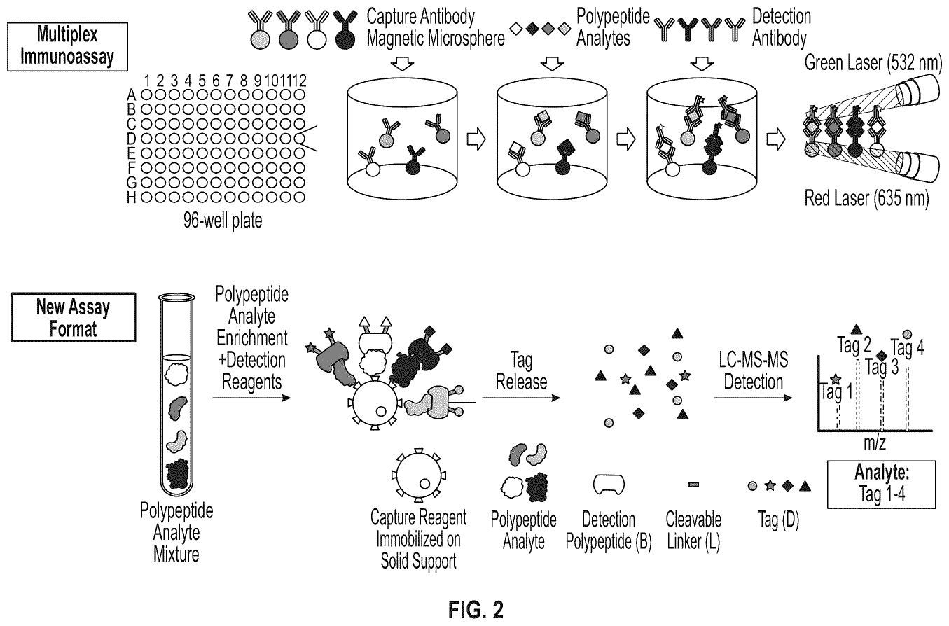

Current LC-MS/MS assays rely on the detection of digested peptide(s) as a surrogate of the total biotherapeutic (FIG. 1, top panel). Bioanalysis (2016) 8:1565-1577; US20120315645(A1). The structure of peptide analyte is limited by the primary amino acid sequence of the biotherapeutic. Thus, the chosen analyte may be used despite not having ideal characteristics for MS detection including; ionization efficiency, peptide stability and tryptic digestion efficiency. The copy number of the analyte is limited by the biotherapeutic—in the case of IgG1, two copies of the surrogate analyte can be produced from one biotherapeutic.

Previous assays and reagents are disclosed in U.S. Pat. Nos. 8,541,178B2, 10,077,318B2, 8,541,178B2, US 2012/0315645A1, US 2017/0315132A1, US 2021/0123928A1, and U.S. Pat. No. 10,077,318. All references cited herein, including patent applications and publications, are incorporated by reference in their entirety.

In some aspects, the present disclosure provides a method for detecting and/or quantitating an analyte (A) in a sample, the method comprising a) affinity enrichment by contacting the sample with a capture reagent to generate a captured polypeptide analyte complex, wherein the capture reagent comprises a polypeptide that binds the polypeptide analyte, b) contacting the captured polypeptide analyte complex with a detection reagent, wherein the detection reagent binds the captured polypeptide analyte complex, wherein the detection reagent is a compound having the formula

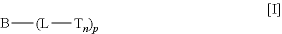

wherein: B is a base detection moiety that binds the polypeptide analyte, L is a cleavable linker, T is a tag suitable for mass spectroscopy, n is an integer greater than or equal to 1, and p is an integer greater than or equal to 1; c) washing the captured polypeptide analyte complex with a buffer to remove unbound detection reagent; d) subjecting the sample from step c) under conditions to cleave the linker to release the tag, and e) analyzing the sample from step d) for presence of the released tag by mass spectroscopy or quantitating the released tags by mass spectroscopy. In some embodiments, each Tag (T) is connected to the base detection moiety (B) via a single linker (L) and at least one Tag is attached to the base detection moiety (p>1). In other embodiments, multiple Tags (T) is connected to the base detection moiety (B) via a single linker (L) (n>1) and one or more Linker-Tags complexes (L-T) are attached to the base detection moiety (p≥1).

In some aspects, the present disclosure provides a method for detecting and/or quantitating a plurality of polypeptide analytes in a sample, the method comprising a) contacting the sample with a plurality of capture reagents to generate a plurality of captured polypeptide analyte complexes, wherein the plurality of capture reagents comprises a plurality of capture polypeptides that bind the plurality of polypeptide analytes, b) contacting the plurality of captured polypeptide analyte complexes with a plurality of detection reagents, wherein the plurality of detection reagents bind the plurality of captured polypeptide analyte complexes, wherein each detection reagent in the plurality of detection reagents is a compound having the formula

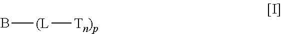

wherein: B is a base detection moiety that binds the polypeptide analyte, L is a cleavable linker, T is a tag suitable for mass spectroscopy, n is an integer greater than or equal to 1, and p is an integer greater than or equal to 1, wherein each tag in the plurality of detection reagents can be distinguished from one another by mass spectroscopy; c) washing the plurality of captured polypeptide analyte complex with a buffer to remove unbound detection reagent; d) subjecting the sample of step c) under conditions to cleave the linker to release the tags from the plurality of detection reagents, e) analyzing the sample of step d) for presence of the plurality of released tags by mass spectroscopy or quantitating the plurality of released tags by mass spectrometry.

In some aspects, the present disclosure provides a detection reagent for use in detecting an analyte in a sample by mass spectroscopy, wherein the detection reagent is a compound having the formula

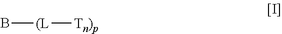

wherein: B is a base detection moiety that binds the polypeptide analyte, L is a cleavable linker, T is a tag suitable for mass spectroscopy, n is an integer greater than or equal to 1, and p is an integer greater than or equal to 1.

### Definitions

“Mass spectrometry” refers to the analytical chemistry technique of identifying an amount and/or type of a compound (e.g., a polypeptide) by measuring the mass-to-charge ratio and abundance of gas-phase ions.

The term “tag”, as used in the present specification, includes a molecule that, by its mass and ionizability, provides an analytically identifiable signal that allows the detection of an analyte. The most commonly used reporter molecules in this type of assay are small molecules and peptides with ionizable basic or acidic polar functional groups with high ionization efficiency and gas-phase fragmentation efficiency, which can be either fluorophores or radionuclide containing molecules (i.e. radioisotopes) and chemiluminescent molecules, unique from the capture reagents or polypeptide analytes. In some embodiments, the tag can be an enzyme or organic dye. In some embodiments, the tags (e.g., small molecules) have adequate MS response (e.g., ˜nM sensitivity) and stability, and do not require additional modification to their structures for MS detection. In some embodiments, a tag disclosed herein can comprise an oligopeptide, for instance, a peptide between about 5 and about 10 amino acids in length. In some embodiments, a tag disclosed herein can comprise a polypeptide comprising no more than 80%, no more than 70%, no more than 60%, no more than 50%, no more than 40%, no more than 30%, no more than 20%, or no more than 10% hydrophobic amino acid content.

The term “polypeptide” or “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component or toxin. Also included within the definition are, for example, polypeptides containing one or more analogs of an amino acid (including, for example, unnatural amino acids, etc.), as well as other modifications known in the art. The terms “polypeptide” and “protein” as used herein specifically encompass antibodies, antibody fragments, growth factor and a domain of receptor proteins.

“Purified” polypeptide (e.g., antibody or immunoadhesin) means that the polypeptide has been increased in purity, such that it exists in a form that is more pure than it exists in its natural environment and/or when initially synthesized and/or amplified under laboratory conditions. Purity is a relative term and does not necessarily mean absolute purity.

The term “antagonist” is used in the broadest sense, and includes any molecule that partially or fully blocks, inhibits, or neutralizes a biological activity of a native polypeptide. In a similar manner, the term “agonist” is used in the broadest sense and includes any molecule that mimics a biological activity of a native polypeptide. Suitable agonist or antagonist molecules specifically include agonist or antagonist antibodies or antibody fragments, fragments, or amino acid sequence variants of native polypeptides, etc. Methods for identifying agonists or antagonists of a polypeptide may comprise contacting a polypeptide with a candidate agonist or antagonist molecule and measuring a detectable change in one or more biological activities normally associated with the polypeptide.

A polypeptide “which binds” an antigen of interest is one that binds the antigen with sufficient affinity such that the polypeptide is useful as a diagnostic and/or therapeutic agent in targeting a cell or tissue expressing the antigen, and does not significantly cross-react with other polypeptides. In such embodiments, the extent of binding of the polypeptide to a “non-target” polypeptide will be less than about 10% of the binding of the polypeptide to its particular target polypeptide as determined by fluorescence activated cell sorting (FACS) analysis or radioimmunoprecipitation (RIA).

With regard to the binding of a polypeptide to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target.

The term “antibody” herein is used in the broadest sense and specifically covers monoclonal antibodies, polyclonal antibodies, multispecific antibodies (e.g. bispecific antibodies including TDB) formed from at least two intact antibodies, and antibody fragments so long as they exhibit the desired biological activity. The term “immunoglobulin” (Ig) is used interchangeably with antibody herein.

Antibodies are naturally occurring immunoglobulin molecules which have varying structures, all based upon the immunoglobulin fold. For example, IgG antibodies have two “heavy” chains and two “light” chains that are disulfide-bonded to form a functional antibody. Each heavy and light chain itself comprises a “constant” (C) and a “variable” (V) region. The V regions determine the antigen binding specificity of the antibody, whilst the C regions provide structural support and function in non-antigen-specific interactions with immune effectors. The antigen binding specificity of an antibody or antigen-binding fragment of an antibody is the ability of an antibody to specifically bind to a particular antigen.

The antigen binding specificity of an antibody is determined by the structural characteristics of the V region. The variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” (HVRs) that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Each V region typically comprises three HVRs, e.g. complementarity determining regions (“CDRs”, each of which contains a “hypervariable loop”), and four framework regions. An antibody binding site, the minimal structural unit required to bind with substantial affinity to a particular desired antigen, will therefore typically include the three CDRs, and at least three, preferably four, framework regions interspersed there between to hold and present the CDRs in the appropriate conformation. Classical four chain antibodies have antigen binding sites which are defined by V_{H} and V_{L} domains in cooperation. Certain antibodies, such as camel and shark antibodies, lack light chains and rely on binding sites formed by heavy chains only. Single domain engineered immunoglobulins can be prepared in which the binding sites are formed by heavy chains or light chains alone, in absence of cooperation between V_{H} and V_{L}.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, MD. (1991)). The constant domains are not involved directly in binding an antibody to an antigen, but exhibit various effector functions, such as participation of the antibody in antibody dependent cellular cytotoxicity (ADCC).

The term “hypervariable region” (HVR) when used herein refers to the amino acid residues of an antibody that are responsible for antigen binding. The hypervariable region may comprise amino acid residues from a “complementarity determining region” or “CDR” (e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

“Framework” or “FR” residues are those variable domain residues other than the hypervariable region residues as herein defined.

“Antibody fragments” comprise a portion of an intact antibody, preferably comprising the antigen binding region thereof. Examples of antibody fragments include Fab, Fab′, F(ab′)₂, and Fv fragments; diabodies; tandem diabodies (taDb), linear antibodies (e.g., U.S. Pat. No. 5,641,870, Example 2; Zapata et al., Protein Eng. 8(10):1057-1062 (1995)); one-armed antibodies, single variable domain antibodies, minibodies, single-chain antibody molecules; multispecific antibodies formed from antibody fragments (e.g., including but not limited to, Db-Fc, taDb-Fc, taDb-CH3, (scFV)4-Fc, di-scFv, bi-scFv, or tandem (di,tri)-scFv); and Bi-specific T-cell engagers (BiTEs).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment that contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called α, δ, ε, γ, and μ, respectively. The subunit structures and three-dimensional configurations of different classes of immunoglobulins are well known.

“Single-chain Fv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains that enables the scFv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “multispecific antibody” is used in the broadest sense and specifically covers an antibody that has polyepitopic specificity. Such multispecific antibodies include, but are not limited to, an antibody comprising a heavy chain variable domain (V_{H}) and a light chain variable domain (V_{L}), where the V_{H}V_{L} unit has polyepitopic specificity, antibodies having two or more V_{L} and V_{H} domains with each V_{H}V_{L} unit binding to a different epitope, antibodies having two or more single variable domains with each single variable domain binding to a different epitope, full length antibodies, antibody fragments such as Fab, Fv, dsFv, scFv, diabodies, bispecific diabodies, triabodies, tri-functional antibodies, antibody fragments that have been linked covalently or non-covalently. “Polyepitopic specificity” refers to the ability to specifically bind to two or more different epitopes on the same or different target(s). “Monospecific” refers to the ability to bind only one epitope. According to one embodiment the multispecific antibody is an IgG antibody that binds to each epitope with an affinity of 5 μM to 0.001 pM, 3 μM to 0.001 pM, 1 μM to 0.001 pM, 0.5 μM to 0.001 pM, or 0.1 μM to 0.001 pM.

The expression “single domain antibodies” (sdAbs) or “single variable domain (SVD) antibodies” generally refers to antibodies in which a single variable domain (VH or VL) can confer antigen binding. In other words, the single variable domain does not need to interact with another variable domain in order to recognize the target antigen. Examples of single domain antibodies include those derived from camelids (lamas and camels) and cartilaginous fish (e.g., nurse sharks) and those derived from recombinant methods from humans and mouse antibodies (Nature (1989) 341:544-546; Dev Comp Immunol (2006) 30:43-56; Trend Biochem Sci (2001) 26:230-235; Trends Biotechnol (2003):21:484-490; WO 2005/035572; WO 03/035694; Febs Lett (1994) 339:285-290; WO00/29004; WO 02/051870).

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the methods provided herein may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

For the purposes herein, an “intact antibody” is one comprising heavy and light variable domains as well as an Fc region. The constant domains may be native sequence constant domains (e.g. human native sequence constant domains) or amino acid sequence variant thereof. Preferably, the intact antibody has one or more effector functions.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

A “naked antibody” is an antibody (as herein defined) that is not conjugated to a heterologous molecule, such as a cytotoxic moiety or radiolabel.

The terms “Fc receptor” or “FcR” are used to describe a receptor that binds to the Fc region of an antibody. In some embodiments, the FcR is a native sequence human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

An “isolated” molecule (e.g., nucleic acid or protein) or cell means it has been identified and separated and/or recovered from a component of its natural environment.

As used herein “essentially the same” indicates that a value or parameter has not been altered by a significant effect. For example, an ionic strength of a chromatography mobile phase at column exit is essentially the same as the initial ionic strength of the mobile phase if the ionic strength has not changed significantly. For example, an ionic strength at column exit that is within 10%, 5% or 1% of the initial ionic strength is essentially the same as the initial ionic strength.

Reference to “about” a value or parameter herein includes (and describes) embodiments that are directed to that value or parameter per se. For example, description referring to “about X” includes description of “X.”

As used herein, the singular form of the articles “a,” “an,” and “the” includes plural references unless indicated otherwise.

It is understood that aspects and embodiments described herein include “comprising,” “consisting,” and/or “consisting essentially of” aspects and embodiments.

### Detection Reagents

In some aspects, the present disclosure provides detections reagent for use in detecting and/or quantitating an analyte in a sample by mass spectroscopy, wherein the detection reagent is a compound having the formula

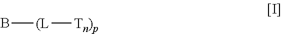

wherein: B is a base detection moiety that binds the analyte, L is a cleavable linker, T is a tag suitable for mass spectroscopy, n is an integer greater than or equal to 1, and p is an integer greater than or equal to 1.

In some embodiments, the base detection moiety of the detection reagent is a polypeptide. In some embodiments, the base detection moiety is an antigen binding protein, an antibody, or a soluble receptor. In some embodiments, the base detection moiety binds the analyte but does not bind any capture reagent used in the methods of the present disclosure.

In some embodiments, n is an integer between 1 and greater than 100. In some embodiments, n is an integer between 1 and 100. In some embodiments, n is an integer between any of 1 and 100, 10 and 100, 20 and 100, 30 and 100, 40 and 100, 50 and 100, 60 and 100, 70 and 100, 80 and 100, 90 and 100, 1 and 90, 10 and 90, 20 and 90, 30 and 90, 40 and 90, 50 and 90, 60 and 90, 70 and 90, 80 and 90, 1 and 80, 10 and 80, 20 and 80, 30 and 80, 40 and 80, 50 and 80, 60 and 80, 70 and 80, 1 and 70, 10 and 70, 20 and 70, 30 and 70, 40 and 70, 50 and 70, 60 and 70, 1 and 60, 10 and 60, 20 and 60, 30 and 60, 40 and 60, 50 and 60, 1 and 50, 10 and 50, 20 and 50, 30 and 50, 40 and 50, 1 and 40, 10 and 40, 20 and 40, 30 and 40, 1 and 30, 10 and 30, 20 and 30, 1 and 20, 10 and 20, or 1 and 10. In some embodiments, n is 1, 2, 3, 4, 5, 6, 7, 8, 9, 10, 11, 12, 13, 14, 15, 16, 17, 18, 19, 20, 25, 30, 40, 50, 60, 70, 75, 80, 90, 100, or greater than 100.

In some embodiments, p is an integer between 1 and greater than 100. In some embodiments, p is an integer between 1 and 100. In some embodiments, p is an integer between any of 1 and 100, 10 and 100, 20 and 100, 30 and 100, 40 and 100, 50 and 100, 60 and 100, 70 and 100, 80 and 100, 90 and 100, 1 and 90, 10 and 90, 20 and 90, 30 and 90, 40 and 90, 50 and 90, 60 and 90, 70 and 90, 80 and 90, 1 and 80, 10 and 80, 20 and 80, 30 and 80, 40 and 80, 50 and 80, 60 and 80, 70 and 80, 1 and 70, 10 and 70, 20 and 70, 30 and 70, 40 and 70, 50 and 70, 60 and 70, 1 and 60, 10 and 60, 20 and 60, 30 and 60, 40 and 60, 50 and 60, 1 and 50, 10 and 50, 20 and 50, 30 and 50, 40 and 50, 1 and 40, 10 and 40, 20 and 40, 30 and 40, 1 and 30, 10 and 30, 20 and 30, 1 and 20, 10 and 20, or 1 and 10. In some embodiments, p is 1, 2, 3, 4, 5, 6, 7, 8, 9, 10, 11, 12, 13, 14, 15, 16, 17, 18, 19, 20, 25, 30, 40, 50, 60, 70, 75, 80, 90, 100, or greater than 100. In some embodiments, p is not 1. In some embodiments, p is not 1 and n is greater than 1. In some embodiments, p is not 1 and n is greater than 2, 3, 4, 5, 6, 7, 8, 9 or 10.

In some embodiments, the linker of the detection reagent is cleavable by an enzyme or by a chemical. In some embodiments, the base detection moiety is not cleaved under the conditions to cleave the linker. In some embodiments, a cleavable linker is a linker with a site of cleavage where a covalent bond is broken to separate tag from a detection polypeptide. Chemically cleavable linkers include but are not limited to acid-cleavable linkers (e.g. hydrazones), photocleavable linkers (e.g. 1-(2-nitrophenyl)ethyl), and reducible linkers (e.g. disulfides). Enzymatic cleavable linkers include at least one specific site for cleavage (e.g., cathepsin B; β-glucuronidase, trypsin, etc.) or includes nonspecific sites for cleavage (e.g., papain, pepsin, proteinase K etc). In some embodiments, the cleavable linker is cleaved by an endopeptidase. In some embodiments, the endopeptidase is papain. A review of cleavable linkers is provided by Bargh, JD et al. Chem Soc Rev, 2019, 48(16):4361-4374.

In some embodiments, the tag of the detection reagent is one or more of a) a molecule that can be ionized and vaporized for analysis by mass spectrometry, b) a molecule with a controlled (e.g., cleavable) conjugation site, c) having a chemical structure distinct from that in the sample, d) provides a good LC-MS response, e) does not compromise binding to the analyte when bound to base, or f) has the capacity to functionalize for a multiplexed assay. In some embodiments, a good LC-MS responses can be achieved by the physicochemical properties of the Tag, including but not limited to high hydrophobicity (log P), high ionizability (pKa) for protonation, and/or a suitable molecular volume (Oss M. et al., Anal Chem 2010, 82(7):2865-2872). In some embodiments, the tag is stable with respect to the sample preparation conditions.

In some embodiments, the tag is made by incorporating stable isotopes into the tag in a manner similar to iTraq (see world wide web.creative-proteomics.com/pdf/Comparison-of-Three-Label-based-Quantification-Techniques-iTRAQ-TMT-and-SILAC.pdf). With this approach, one tag may be functionalized to multiple tags that give different m/z responses and can be separated by LC-MS/MS.

In some embodiments, the detection reagent and the capture reagent bind the target analyte at a different site; for example, in use of the methods of the present disclosure.

In some embodiments, the base detection moiety (of the detection reagent) and the binding reagent (of the capture reagent) can be identical or different. For example, in an embodiment where the base detection moiety and the binding reagent are antigen binding proteins such as antibodies, the antigen binding proteins can be the same or different. For instance, the base detection moiety and the binding reagent can be the same antibody or antibody fragment, each binding to separate copies of the same epitope on the target analyte. For instance, the base detection moiety and the binding reagent can be different antibodies or antibody fragments that bind to different epitopes on the target analyte. In some embodiments, the present disclosure provides a plurality of detection reagents for detecting and/or quantitating a plurality of analytes in a sample. In some embodiments, each tag in the plurality of detection reagents can be distinguished from one another by mass spectroscopy. In some embodiments, the present disclosure provides a library of detection reagents, wherein the library of detection reagents comprises a plurality of detection reagents for detecting and/or quantitating a plurality of analytes in a sample. In some embodiments, each tag in the plurality of detection reagents or the library can be distinguished by mass spectroscopy. In some embodiments, the present disclosure provides a library of detection reagents, wherein the library of detection reagents comprises any suitable detection reagents, such as binding proteins, antibodies, ligands, or soluble receptors. In some embodiments, the plurality of analytes is two or more of therapeutic polypeptides, therapeutic antibodies, and/or biomarkers.

### Linkers

A linker, with respect to the detection reagents of the present disclosure, is a bifunctional or multifunctional chemical moiety comprising a covalent bond or a chain of atoms that covalently attaches the Tag to the base detection moiety.

Peptide linkers. In some embodiments, the linker is a peptide with an amino acid sequence of two or more amino acids in length. The linker can consist of neutral, polar, or nonpolar amino acids. A linker can be, for example, 2 to 100 amino acids in length, such as between 2 and 50 amino acids in length, for example, 3, 5, 10, 15, 20, 25, 30, 35, 40, 45, or 50 amino acids in length. A linker can be “cleavable,” for example, by auto-cleavage, or enzymatic or chemical cleavage. Cleavage sites in amino acid sequences and enzymes and chemicals that cleave at such sites are well known in the art and are also described herein.

In some embodiments, enzymatic cleavage of a peptide linker involves the use of an endopeptidase such as, for example, Lys-C, Lys-N, Asp-N, Arg-C, AsnC, V8, Glu-C, chymotrypsin, trypsin, pepsin, papain, cathepsin B, thrombin, PNGaseF, Genenase, Factor Xa, TEV (tobacco etch virus cysteine protease), Enterokinase, HRV C3 (human rhinovirus C3 protease), Kininogenase, as well as subtilisin-like proprotein convertases (e.g., Furin (PC1), PC2, or PC3) or N-arginine dibasic convertase. Chemical cleavage may involve use of, for example, hydroxylamine, N-chlorosuccinimide, N-bromosuccinimide, or cyanogen bromide.

Chemical linkers. In some embodiments, the Linker may have an electrophilic group reactive with a nucleophilic group present on the base detection moiety, such as thiol or amino. A cysteine thiol of the base detection moiety is reactive with an electrophilic group on a Linker and forms a covalent bond to a Linker. Useful electrophilic groups include, but are not limited to, maleimide and haloacetamide groups. Linkers also include a divalent radical such as an alkyldiyl, an arylene, a heteroarylene, moieties such as: −(CR₂)ₙO(CR₂)ₙ—, repeating units of alkyloxy (e.g. polyethylenoxy, PEG, polymethyleneoxy) and alkylamino (e.g. polyethyleneamino, Jeffamine™); and diacid ester and amides including succinate, succinamide, diglycolate, malonate, and caproamide. Useful nucleophilic groups on an antibody include but are not limited to, sulfhydryl, hydroxyl, and amino groups.

In another embodiment, a linker reagent or drug-linker reagent has a reactive nucleophilic functional group which is reactive with an electrophile present on a base detection moiety to form a covalent bond. Useful electrophilic groups on an antibody include, but are not limited to, aldehyde and ketone carbonyl groups. Useful nucleophilic groups on a linker include, but are not limited to, hydrazide, oxime, amino, hydrazine, thiosemicarbazone, hydrazine carboxylate, and arylhydrazide.

The linker may be composed of one or more linker components. Exemplary linker components include 6-maleimidocaproyl (“MC”), maleimidopropanoyl (“MP”), valine-citrulline (“val-cit” or “vc”), alanine-phenylalanine (“ala-phe” or “af”), p-aminobenzyloxycarbonyl (“PAB”), N-succinimidyl 4-(2-pyridylthio) pentanoate (“SPP”), N-succinimidyl 4-(N-maleimidomethyl)cyclohexane-1 carboxylate (“SMCC”), N-Succinimidyl (4-iodo-acetyl)aminobenzoate (“SIAB”), ethyleneoxy —CH₂CH₂O— as one or more repeating units (“EO” or “PEO”).

In some embodiments, the base detection moiety has one or more lysine residues that can be chemically modified to introduce one or more sulfhydryl groups. The antibody unit bonds to the Linker unit via the sulfhydryl group's sulfur atom. The reagents that can be used to modify lysines include, but are not limited to, N-succinimidyl S-acetylthioacetate (SATA) and 2-Iminothiolane hydrochloride (Traut's Reagent).

In some embodiments, the antibody can have one or more carbohydrate groups that can be chemically modified to have one or more sulfhydryl groups. The antibody unit bonds to the linker, such as the Stretcher Unit, via the sulfhydryl group's sulfur atom. In yet another embodiment, the antibody can have one or more carbohydrate groups that can be oxidized to provide an aldehyde (—CHO) group (see for example, Laguzza, et al. (1989) J. Med. Chem. 32(3):548-55). The corresponding aldehyde can form a bond with a Reactive Site on a Stretcher. Reactive sites on a Stretcher that can react with a carbonyl group on an antibody include, but are not limited to, hydrazine and hydroxylamine. Other protocols for the modification of proteins for the attachment or association of Drug Units are described in Coligan et al., “Current Protocols in Protein Science”, vol. 2, John Wiley & Sons (2002), incorporated herein by reference.

In some embodiments, the linker may be substituted with groups which modulated solubility or reactivity. For example, a charged substituent such as sulfonate (—SO₃—) or ammonium, may increase water solubility of the reagent and facilitate the coupling reaction of the linker reagent with the antibody or the drug moiety, or facilitate the coupling reaction of Ab-L (antibody-linker) with D, or D-L (drug linker reagent) with Ab, depending on the synthetic route employed to prepare the detection reagent.

The compounds of the present disclosure expressly contemplate, but are not limited to, detection reagents prepared with cross-linker reagents: BMPEO, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate), and including bis-maleimide reagents: DTME, BMB, BMDB, BMH, BMOE, BM(PEO)₂, and BM(PEO)₃, which are commercially available from Pierce Biotechnology, Inc. Bis-maleimide reagents allow the attachment of the thiol group of a cysteine residue of an antibody to a thiol-containing drug moiety, label, or linker intermediate, in a sequential or concurrent fashion. Other functional groups besides maleimide, which are reactive with a thiol group of an antibody, drug moiety, label, or linker intermediate include iodoacetamide, bromoacetamide, vinyl pyridine, disulfide, pyridyl disulfide, isocyanate, and isothiocyanate. Useful linker reagents can also be obtained via other commercial sources, such as Molecular Biosciences Inc. (Boulder, Colo.), or synthesized in accordance with procedures described in Toki et al (2002) J. Org. Chem. 67:1866-1872; U.S. Pat. No. 6,214,345 to Firestone et al; WO 02/088172; US 2003130189; US 2003096743; WO 03/026577; WO 03/043583; and WO 04/032828.

Reactive thiol groups of cysteine engineered antibodies (US 2007/0092940) react with linker reagents or drug-linker intermediates, with electrophilic functional groups such as maleimide or a-halo carbonyl, according to the conjugation method at page 766 of Klussman, et al (2004), Bioconjugate Chemistry 15(4):765-773.

### Methods

In some aspects, the present disclosure provides a method for detecting an analyte (A) in a sample, the method comprising a) affinity enrichment by contacting the sample with a capture reagent to generate a captured analyte complex, wherein the capture reagent comprises a polypeptide that binds the analyte, b) contacting the captured analyte complex with a detection reagent, wherein the detection reagent binds the captured analyte complex, wherein the detection reagent is a compound having the formula

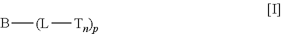

wherein: B is a base detection moiety that binds the analyte, L is a cleavable linker, T is a tag suitable for mass spectroscopy, n is an integer greater than or equal to 1, and p is an integer greater than or equal to 1; c) washing the captured analyte complex with a buffer to remove unbound detection reagent; d) subjecting the sample from step c) under conditions to cleave the linker to release the tag, and e) quantifying the released tag by mass spectroscopy.

In some aspects, the present disclosure provides a method for quantitating an analyte (A) in a sample, the method comprising a) affinity enrichment by contacting the sample with a capture reagent to generate a captured analyte complex, wherein the capture reagent comprises a polypeptide that binds the analyte, b) contacting the captured analyte complex with a detection reagent, wherein the detection reagent binds the captured analyte complex, wherein the detection reagent is a compound having the formula

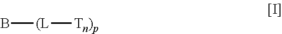

wherein: B is a base detection moiety that binds the analyte, L is a cleavable linker, T is a tag suitable for mass spectroscopy, n is an integer greater than or equal to 1, and p is an integer greater than or equal to 1; c) washing the captured analyte complex with a buffer to remove unbound detection reagent; d) subjecting the sample from step c) under conditions to cleave the linker to release the tag, and e) quantifying the released tag by mass spectroscopy. In some embodiments, each Tag (T) is connected to the base detection moiety (B) via a single linker (L) and at least one Tag (n≥1) is attached to the base detection moiety. In other embodiments, multiple Tags (T) are connected to the base detection moiety (B) via a single linker (L) (n>1) and one or more Linker-Tags complexes (L-T) are attached to the base detection moiety (p≥1).

In some aspects, the present disclosure provides a method for detecting a plurality of analytes in a sample, the method comprising a) contacting the sample with a plurality of capture reagents to generate a plurality of captured analyte complexes, wherein the plurality of capture reagents comprises a plurality of capture polypeptides that bind the plurality of analytes, b) contacting the plurality of captured analyte complexes with a plurality of detection reagents, wherein the plurality of detection reagents bind the plurality of captured analyte complexes, wherein each detection reagent in the plurality of detection reagents is a compound having the formula

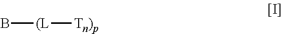

wherein: B is a base detection moiety that binds the analyte, L is a cleavable linker, T is a tag suitable for mass spectroscopy, n is an integer greater than or equal to 1, and p is an integer greater than or equal to 1, wherein each tag in the plurality of detection reagents can be distinguished from one another by mass spectroscopy; c) washing the plurality of captured analyte complex with a buffer to remove unbound detection reagent; d) subjecting the sample from step c) under conditions to cleave the linker to release the tags from the plurality of detection reagents, e) analyzing the sample from step d) for presence of the plurality of released tags by mass spectroscopy.

In some aspects, the present disclosure provides a method for quantitating a plurality of analytes in a sample, the method comprising a) contacting the sample with a plurality of capture reagents to generate a plurality of captured analyte complexes, wherein the plurality of capture reagents comprises a plurality of capture polypeptides that bind the plurality of analytes, b) contacting the plurality of captured analyte complexes with a plurality of detection reagents, wherein the plurality of detection reagents bind the plurality of captured analyte complexes, wherein each detection reagent in the plurality of detection reagents is a compound having the formula

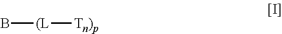

wherein: B is a base detection moiety that binds the analyte, L is a cleavable linker, T is a tag suitable for mass spectroscopy, n is an integer greater than or equal to 1, and p is an integer greater than or equal to 1, wherein each tag in the plurality of detection reagents can be distinguished from one another by mass spectroscopy; c) washing the plurality of captured analyte complex with a buffer to remove unbound detection reagent; d) subjecting the sample of step c) under conditions to cleave the linker to release the tags from the plurality of detection reagents, e) quantifying the plurality of released tags by mass spectroscopy.

In some embodiments, the base detection moiety of the detection reagent is an antigen binding protein, an antibody, or a soluble receptor. In some embodiments, the base detection moiety binds the analyte but does not bind any capture reagent used in the methods of the present disclosure.

In some embodiments, n is an integer between 1 and greater than 100. In some embodiments, n is an integer between 1 and 100. In some embodiments, n is an integer between any of 1 and 100, 10 and 100, 20 and 100, 30 and 100, 40 and 100, 50 and 100, 60 and 100, 70 and 100, 80 and 100, 90 and 100, 1 and 90, 10 and 90, 20 and 90, 30 and 90, 40 and 90, 50 and 90, 60 and 90, 70 and 90, 80 and 90, 1 and 80, 10 and 80, 20 and 80, 30 and 80, 40 and 80, 50 and 80, 60 and 80, 70 and 80, 1 and 70, 10 and 70, 20 and 70, 30 and 70, 40 and 70, 50 and 70, 60 and 70, 1 and 60, 10 and 60, 20 and 60, 30 and 60, 40 and 60, 50 and 60, 1 and 50, 10 and 50, 20 and 50, 30 and 50, 40 and 50, 1 and 40, 10 and 40, 20 and 40, 30 and 40, 1 and 30, 10 and 30, 20 and 30, 1 and 20, 10 and 20, or 1 and 10. In some embodiments, n is 1, 2, 3, 4, 5, 6, 7, 8, 9, 10, 11, 12, 13, 14, 15, 16, 17, 18, 19, 20, 25, 30, 40, 50, 60, 70, 75, 80, 90, 100, or greater than 100.

In some embodiments, p is an integer between 1 and greater than 100. In some embodiments, p is an integer between 1 and 100. In some embodiments, p is an integer between any of 1 and 100, 10 and 100, 20 and 100, 30 and 100, 40 and 100, 50 and 100, 60 and 100, 70 and 100, 80 and 100, 90 and 100, 1 and 90, 10 and 90, 20 and 90, 30 and 90, 40 and 90, 50 and 90, 60 and 90, 70 and 90, 80 and 90, 1 and 80, 10 and 80, 20 and 80, 30 and 80, 40 and 80, 50 and 80, 60 and 80, 70 and 80, 1 and 70, 10 and 70, 20 and 70, 30 and 70, 40 and 70, 50 and 70, 60 and 70, 1 and 60, 10 and 60, 20 and 60, 30 and 60, 40 and 60, 50 and 60, 1 and 50, 10 and 50, 20 and 50, 30 and 50, 40 and 50, 1 and 40, 10 and 40, 20 and 40, 30 and 40, 1 and 30, 10 and 30, 20 and 30, 1 and 20, 10 and 20, or 1 and 10. In some embodiments, p is 1, 2, 3, 4, 5, 6, 7, 8, 9, 10, 11, 12, 13, 14, 15, 16, 17, 18, 19, 20, 25, 30, 40, 50, 60, 70, 75, 80, 90, 100, or greater than 100. In some embodiments, p is not 1. In some embodiments, p is not 1 and n is greater than 1. In some embodiments, p is not 1 and n is greater than 2, 3, 4, 5, 6, 7, 8, 9 or 10.

In some embodiments, the linker of the detection reagent is cleavable by an enzyme or by a chemical. In some embodiments, the base detection moiety is not cleaved under the conditions to cleave the linker. In some embodiments, a cleavable linker is a linker with a site of cleavage where a covalent bond is broken to separate tag from a detection polypeptide. Chemically cleavable linkers include but are not limited to acid-cleavable linkers (e.g. hydrazones) and reducible linkers (e.g. disulfides). Enzymatic cleavable linkers include at least one specific site for cleavage (e.g., cathepsin B; β-glucuronidase, trypsin, etc.) or includes nonspecific sites for cleavage (e.g., papain, pepsin, proteinase K etc). In some embodiments, the cleavable linker is cleaved by an endopeptidase. In some embodiments, the cleavable linker is cleaved by an irradiation with near-UV light. In some embodiments, the endopeptidase is papain. A review of cleavable linkers is provided by Bargh, JD et al. Chem Soc Rev, 2019, 48(16):4361-4374.

In some embodiments, the tag of the detection reagent is one or more of a) a molecule that can be ionized and vaporized for analysis by mass spectrometry, b) a molecule with a controlled conjugation site, c) unique from other peptides in the sample, d) provides a good LC-MS response, e) does not compromise binding to the analyte when bound to base, or f) has the capacity to functionalize for a multiplexed assay. In some embodiments, a good LC-MS responses can be achieved by the physicochemical properties of the Tag, including but not limited to high hydrophobicity (log P), high ionizability (pKa) for protonation, and/or a suitable molecular volume (Oss M. et al., Anal Chem 2010, 82(7):2865-2872). In some embodiments, the tag is stable with respect to the sample preparation conditions.

In some embodiments, the tag is made by incorporating stable isotopes into the tag in a manner similar to iTraq (see world wide web.creative-proteomics.com/pdf/Comparison-of-Three-Label-based-Quantification-Techniques-iTRAQ-TMT-and-SILAC.pdf). With this approach, one tag may be functionalized to multiple tags that give different m/z responses and can be separated by LC-MS/MS.

In some embodiments, the detection reagent binds the target analyte at a different site than a capture polypeptide; for example, in use in the methods of the present disclosure.

In some embodiments, the present disclosure provides a plurality of detection reagents for detecting and/or quantitating a plurality of analytes in a sample. In some embodiments, each tag in the plurality of detection reagents can be distinguished from one another by mass spectroscopy. In some embodiments, the present disclosure provides a library of detection reagents, wherein the library of detection reagents comprises a plurality of detection reagents for detecting and/or quantitating a plurality of analytes in a sample. In some embodiments, each tag in the plurality of detection reagents or the library can be distinguished by mass spectroscopy. In some embodiments, the present disclosure provides a library of detection reagents, wherein the library of detection reagents comprises. In some embodiments, the plurality of analytes is two or more of therapeutic polypeptides, therapeutic antibodies, and/or biomarkers.

In some embodiments, the capture reagent is an antigen binding protein, an antibody, a soluble receptor, a protein A, a protein G, a protein L, or an extracellular domain. In some embodiments, the capture agent is immobilized on a solid support. In some embodiments, the solid support is a bead (e.g., a magnetic bead) or resin (e.g., agarose resin). In some embodiments, the solid support is polyvinylidene fluoride or nitrocellulose membrane. In some embodiments, the capture reagent is in a buffer. In some embodiments, the capture reagent is in phosphate buffered saline (PBS), HEPES, Tris phosphate, 3-((3-cholamidopropyl) dimethylammonio)-1-propanesulfonate (i.e., CHAPS), ethylenediaminetetraacetic acid (EDTA), or polysorbate (e.g., Tween). In some embodiments, the buffer contains bovine serum albumin (BSA). In some embodiments, the buffer comprises between about 0.1% BSA and about 1% BSA. In some embodiments, the buffer comprises any of about 0.1% BSA, 0.2% BSA, 0.3% BSA, 0.4% BSA, 0.5% BSA, 0.6% BSA, 0.7% BSA, 0.8% BSA, 0.9% BSA, 1.0% BSA. In some embodiments, the buffer is PBS and comprises any of about 0.1% BSA, 0.2% BSA, 0.3% BSA, 0.4% BSA, 0.5% BSA, 0.6% BSA, 0.7% BSA, 0.8% BSA, 0.9% BSA, 1.0% BSA.

In some embodiments of the present disclosure, affinity enrichment is used to capture the analyte. In some embodiments, the affinity enrichment is an immunoaffinity enrichment. In some embodiments, the affinity enrichment is conducted using a capture reagent immobilized on a solid support. In some embodiments, the solid support is a bead (e.g., a magnetic bead). In some embodiments, the solid support comprises Dynabeads®. In some embodiments, the solid support comprises streptavidin-coated Dynabeads®. In some embodiments, the capture reagent comprises biotin. In some embodiments, the capture reagent comprises biotin and the solid support comprises streptavidin.

In some embodiments, the capture reagent is incubated with the immobilized support; for example for any of about 15 minutes, 30 minutes, 45 minutes, 1 hour or more than 1 hour. In some embodiments, the capture reagent is incubated with the immobilized support at about 4° C., room temperature, 25° C., 37° C. or less. In some embodiments, the beads are washed one, two, three, four, five or more than five times to remove non-bound capture reagent from the system.

In some embodiments, the complex of the immobilized capture reagent is incubated with the sample comprising the analyte (or control); for example, for any of about 15 minutes, 30 minutes, 45 minutes, 1 hour or more than 1 hour. In some embodiments, complex of the immobilized capture reagent and the sample (or control) is incubated at about 4° C., room temperature, 25° C., 37° C. or less. In some embodiments, the complex of the immobilized capture reagent and the sample (or control) is washed one, two, three, four, five or more than five times to remove non-bound components of sample or control from the system.

In some embodiments, the complex of the immobilized capture reagent and sample (or control) is incubated with the detection reagent; for example for any of about 15 minutes, 30 minutes, 45 minutes, 1 hour or more than 1 hour. In some embodiments, the complex of the immobilized capture reagent and sample (or control) is incubated at about 4° C., room temperature, 25° C., 37° C. or less.

In some embodiments, the complex of the immobilized capture reagent, the sample (or control), and the detection reagent is washed one, two, three, four, five or more than five times to remove non-bound detection reagent from the system. In some embodiments, the immobilized capture reagent, the sample (or control), and the detection reagent are incubated at about 60° C. for about 60 minutes to reduce disulfide bonds and for thermal denaturation followed by alkylation by iodoacetamide or iodoacetic acid. Following this, a cleavage reagent (e.g., trypsin) is then added to each complex to release the tag. In some embodiments, the immobilized capture reagent, the sample (or control), and the detection reagent are incubated with a cleavage reagent (e.g., activated papain) at about 25° C. for about 60 minutes to release the tag. In some embodiments, the complex of the immobilized capture reagent, the sample (or control), and the detection reagent or the released tag is evaporated to dryness (e.g., with nitrogen at 45° C.). In some embodiments, the evaporated complexes are resuspended in an organic solvent (e.g., 80/20 H₂O/MeCN), followed by an organic solvent (e.g., MeOH) for protein precipitation. In some embodiments, the resuspended complex or released tag is then analyzed using LC-MS/MS.

In a non-limiting example, LC-MS/MS is carried out using a Shimadzu (Tokyo, Japan) Nexera HPLC system interfaced to a SCIEX QTRAP® 5500 (AB Sciex, Concord, ON). In some embodiments, the released tag analyte is analyzed using a Phenomenex Synergi RP-Max for chromatographic separation. Positive ion electrospray mass spectrometry in multiple reaction monitoring (MRM) mode is then used for the detection of analyte. In some embodiments, the source temperature and turbo ionspray voltage were set to about 525° C. and about 5000 V, respectively. In some embodiments, optimized collision energy is about 100 eV. In some embodiments, for the analysis of released analyte, the transitions of m/z 718.4→152.1 and m/z 726.6→152.1 are monitored. In some embodiments, for the analysis of released analyte (e.g., polypeptide analyte), the transitions of m/z 555.8→397.2, m/z 938.0→836.7, and m/z 941.5→840.0 are monitored. The collision energy conditions and transitions to be monitored for each tag can be determined for any particular analyte/tag combination. In some embodiments, a tag disclosed herein can have a value of transition ranging from 0-2000 m/z→0-2000 m/z, and the transition values can be different for different tags. In some embodiments, a tag disclosed herein can provide a good LC-MS response monitorable by a precursor ion-product ion pair ranging from 0-2000 m/z.

In some embodiments, the detection reagents of the present disclosure are useful for detecting the presence of an analyte in a biological sample. The term “detecting” as used herein encompasses quantitative or qualitative detection. In certain embodiments, a biological sample comprises blood, plasma, serum, cells, urine, aqueous humor, vitreous humor, tears, or tissue. In certain embodiments, the method comprises contacting the biological sample with a detection reagent as described herein under conditions permissive for binding of the detection reagent to the analyte, and detecting whether a complex is formed between the detection reagent and the analyte. Such a method may be an in vitro or in vivo method.

Labeled and conjugated detection reagents are utilized in certain embodiments of the methods of the present disclosure. Labels include, but are not limited to, labels or moieties that are detected directly (such as fluorescent, chromophore, electron-dense, chemiluminescent, and radioactive labels), as well as moieties, such as enzymes or ligands, which are detected indirectly, e.g., through an enzymatic reaction or molecular interaction. Exemplary labels include, but are not limited to, the radioisotopes ³²p, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, 13-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

In some embodiments, a signal-to-noise ratio (S/N) in detection using a method based on IA-LC-MS/MS disclosed herein is greater than about 3, greater than about 4, greater than about 5, greater than about 6, greater than about 7, greater than about 8, greater than about 9, greater than about 10, greater than about 11, greater than about 12, greater than about 13, greater than about 14, greater than about 15, greater than about 16, greater than about 17, greater than about 18, greater than about 19, or greater than about 20. In some embodiments, a LLOQ (limit of quantitation) in detection using a method based on IA-LC-MS/MS disclosed herein is no more than 25, no more than 20, no more than 15, no more than 10, no more than 5, or no more than 1 ng/mL. In some embodiments, a LLOQ (limit of quantitation) in detection using a method based on IA-LC-MS/MS disclosed herein is between about 0.1 and about 20 ng/ml, between about 0.2 and about 15 ng/mL, or between about 0.5 and about 10 ng/ml. In some embodiments, a method based on IA-LC-MS/MS disclosed herein achieves high detection precision and accuracy (e.g., Coefficients of Variants (CV)), S/N, magnitude of signal response compared to a blank sample, and/or correlation coefficient that indicates how independents variable (e.g., analyte concentrations) can be extrapolated from the known concentrations of the standards.

### Analytes

Examples of analytes to be detected and/or quantified using the methods of the present disclosure include but are not limited to nucleic acid analytes or polypeptide analytes. In some embodiments, a polypeptide analyte can be an immunoglobulins, immunoadhesins, antibodies, enzymes, hormones, fusion proteins, Fc-containing proteins, immunoconjugates, cytokines and interleukins. In some embodiments, the polypeptide analytes include, but are not limited to, mammalian proteins, such as, e.g., renin; a hormone; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrand factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and-beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; an enzyme; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; Protein A or D; rheumatoid factors; a neurotrophic factor such as bone-derived neurotrophic factor (BDNF), neurotrophin-3,-4,-5, or-6 (NT-3, NT-4, NT-5, or NT-6), or a nerve growth factor such as NGF-b; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and-II (IGF-I and IGF-II); des(1-3)-IGF-I (brain IGF-I), insulin-like growth factor binding proteins (IGFBPs); a cytokine; CD proteins such as CD3, CD4, CD8, CD19 and CD20; erythropoietin; osteoinductive factors; immunotoxins; a fusion polypeptide, i.e. a polypeptide comprised on two or more heterologous polypeptides or fragments thereof and encoded by a recombinant nucleic acid; an Fc-containing polypeptide, for example, a fusion protein comprising an immunoglobulin Fc region, or fragment thereof, fused to a second polypeptide; an immunoconjugate; a bone morphogenetic protein (BMP); an interferon such as interferon-alpha,-beta, and-gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; a tumor associated antigen such as CA125 (ovarian cancer antigen) or HER2, HER3 or HER4 receptor; immunoadhesins; and fragments and/or variants of any of the above-listed proteins as well as antibodies, including antibody fragments, binding to a protein, including, for example, any of the above-listed proteins.

Examples of analytes to be detected and/or quantified using the methods of the present disclosure include but are not limited to peptides/proteins conjugated to one or more biocompatible polymers (e.g. polyethylene glycol or hyaluronic acid).

### Antibodies

In some embodiments of any of the methods described herein, the polypeptide analyte described herein is an antibody.

Molecular targets for antibodies include CD proteins and their ligands, such as, but not limited to: (i) CD3, CD4, CD8, CD19, CD11a, CD20, CD22, CD34, CD40, CD79a (CD79a), and CD79β (CD79b); (ii) members of the ErbB receptor family such as the EGF receptor, HER2, HER3 or HER4 receptor; (iii) cell adhesion molecules such as LFA-1, Mac1, p150,95, VLA-4, ICAM-1, VCAM and av/β3 integrin, including either alpha or beta subunits thereof (e.g., anti-CD11a, anti-CD18 or anti-CD11b antibodies); (iv) growth factors such as VEGF; IgE; blood group antigens; flk2/flt3 receptor; obesity (OB) receptor; mp/receptor; CTLA-4; protein C, BR3, c-met, tissue factor, β7, etc.; and (v) cell surface and transmembrane tumor-associated antigens (TAA), such as those described in U.S. Pat. No. 7,521,541, incorporated herein by reference in its entirety.

Other exemplary antibodies include those selected from, and without limitation, anti-CD20 antibody, anti-CD40 antibody, anti-HER2 antibody, anti-IL6 antibody, anti-IgE antibody, anti-IL13 antibody, anti-Flu A antibody, anti-TIGIT antibody, anti-PD-L1 antibody, anti-VEGF-A antibody, anti-VEGF-A/ANG2 antibody, anti-CD79b antibody, anti-ST2 antibody, anti-factor D antibody, anti-factor IX antibody, anti-factor X antibody, anti-abeta antibody, anti-tau antibody, anti-CEA antibody, anti-CEA/CD3 antibody, anti-CD20/CD3 antibody, anti-FcRH5/CD3 antibody, anti-Her2/CD3 antibody, anti-FGFR1/KLB antibody, a FAP-4-1 BBL fusion protein, a FAP-IL2v fusion protein, anti-estrogen receptor antibody, anti-progesterone receptor antibody, anti-p53 antibody, anti-EGFR antibody, anti-cathepsin D antibody, anti-Bcl-2 antibody, anti-E-cadherin antibody, anti-CA125 antibody, anti-CA15-3 antibody, anti-CA19-9 antibody, anti-c-erbB-2 antibody, anti-P-glycoprotein antibody, anti-CEA antibody, anti-retinoblastoma protein antibody, anti-ras oncoprotein antibody, anti-Lewis X antibody, anti-Ki-67 antibody, anti-PCNA antibody, anti-CD3 antibody, anti-CD4 antibody, anti-CD5 antibody, anti-CD7 antibody, anti-CD8 antibody, anti-CD9/p24 antibody, anti-CD10 antibody, anti-CD11a antibody, anti-CD11c antibody, anti-CD13 antibody, anti-CD14 antibody, anti-CD15 antibody, anti-CD19 antibody, anti-CD22 antibody, anti-CD23 antibody, anti-CD30 antibody, anti-CD31 antibody, anti-CD33 antibody, anti-CD34 antibody, anti-CD35 antibody, anti-CD38 antibody, anti-CD41 antibody, anti-LCA/CD45 antibody, anti-CD45RO antibody, anti-CD45RA antibody, anti-CD39 antibody, anti-CD100 antibody, anti-CD95/Fas antibody, anti-CD99 antibody, anti-CD106 antibody, anti-ubiquitin antibody, anti-CD71 antibody, anti-c-myc antibody, anti-cytokeratin antibody, anti-vimentins antibody, anti-HPV proteins antibody, anti-kappa light chains antibody, anti-lambda light chains antibody, anti-melanosomes melanosomes antibody, anti-prostate specific antigen antibody, anti-S-100 antibody, anti-tau antigen antibody, anti-fibrin antibody, anti-keratins antibody and anti-Tn-antigen antibody.

### Capture Reagents

In some embodiments, the capture reagent of the present disclosure comprises a binding reagent. An example of a binding reagent includes but is not limited to a polypeptide, an antigen binding protein, an antibody, a soluble receptor, a protein A, a protein G, or a protein L. In some embodiments, the binding agent is immobilized on a solid support.

A solid support refers to any material that is appropriate for or can be modified to be appropriate for the attachment of a capture reagent of the present disclosure. Examples of solid supports for use in the methods disclosed herein include but are not limited to glass and modified or functionalized glass, plastics (including acrylics, polystyrene, methylstyrene, polyurethanes, Teflon™, etc.), paramagnetic materials, thoria sol, carbon graphite, titanium oxide, latex or cross-linked dextrans such as Sepharose, cellulose polysaccharides, nylon or nitrocellulose, ceramics, resins, silica or silica-based materials including silicon and modified silicon, carbon metals, inorganic glasses, optical fiber bundles, and a variety of other polymers. In some embodiments, the solid supports can be located in microtiter well plates (e.g., a 96-well, 384-well or 1536-well plate). In some embodiments, the solid supports can be located within a flow cell or flow cell apparatus (e.g., a flow cell on a Biacore™ chip or a protein chip).

In some embodiments, the solid support can be a resin (e.g., agarose resin). In some embodiments, the solid support can be polyvinylidene fluoride or nitrocellulose membrane. In some embodiments, the solid support can be a bead (e.g., a magnetic bead), microsphere, particle, membrane, chip, slide, well, and test tube. Beads include microspheres or particles. By “microspheres” or “particles” or grammatical equivalents herein is meant small, discrete, non-planar particles in the micrometer or nanometer dimensions. In some embodiments the bead can be spherical, in other embodiments the bead is irregular. Alternatively or additionally, the beads can be porous. The bead sizes range from nanometers to millimeters with beads from about 0.2 to about 200 microns being preferred in some embodiments. In other embodiments, bead size can range from about 0.5 to about 5 microns. In some embodiments, beads smaller than 0.2 microns and larger than 200 microns can be used. In some embodiments, the solid support can include an array of wells or depressions in a surface. This can be fabricated as is known in the art using a variety of techniques, including, photolithography, stamping techniques, molding techniques and microetching techniques. As will be appreciated by those skilled in the art, the technique used will depend on the composition and shape of the array substrate.

### Kits and Articles of Manufacture

In some aspects of the present disclosure, a kit or article of manufacture is provided for methods to detect and/or quantify one or more analytes in a sample. In some embodiments, the kit further comprises a container which holds reference analytes. In some embodiments, the kit further comprises a container or surface for immobilizing a capture reagent. In some embodiments, the kit further comprises detection reagents (e.g., a base detection moiety comprising one or more tags attached by a cleavable linker.

The containers hold the formulations and the labels on, or associated with, the containers may indicate directions for use. The article of manufacture may further include other materials desirable from a commercial and user standpoint, including other buffers, diluents, cultureware, reagents for detecting reporter molecules, and package inserts with instructions for use.

### Exemplary Embodiments

Among the provided embodiments are:
1. A method for detecting an analyte (A) in a sample, the method comprising
   a) affinity enrichment by contacting the sample with a capture reagent to generate a captured analyte complex, wherein the capture reagent comprises a binding reagent that binds the analyte,
   b) contacting the captured analyte complex with a detection reagent, wherein the detection reagent binds the captured analyte complex, wherein the detection reagent is a compound having the formula

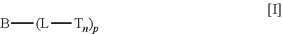

wherein:
   B is a base detection moiety that binds the analyte,
   L is a cleavable linker,
   T is a tag suitable for mass spectroscopy,
   n is an integer greater than or equal to 1, and
   p is an integer greater than or equal to 1;
   c) washing the captured analyte complex with a buffer to remove unbound detection reagent;
   d) subjecting the sample from step c) under conditions to cleave the linker to release the tag, and
   e) analyzing the sample from step d) for presence of the released tag by mass spectroscopy.
2. A method for quantifying an analyte (A) in a sample, the method comprising
   a) affinity enrichment by contacting the sample with a capture reagent to generate a captured analyte complex, wherein the capture reagent comprises a binding reagent that binds the analyte,
   b) contacting the captured analyte complex with a detection reagent, wherein the detection reagent binds the captured analyte complex, wherein the detection reagent is a compound having the formula

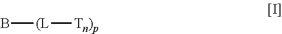

wherein:
   B is a base detection moiety that binds the analyte,
   L is a cleavable linker,
   T is a tag suitable for mass spectroscopy,
   n is an integer greater than or equal to 1, and
   p is an integer greater than or equal to 1;
   c) washing the captured analyte complex with a buffer to remove unbound detection reagent;
   d) subjecting the sample from step c) under conditions to cleave the linker to release the tag, and
   e) quantifying the released tag by mass spectroscopy.
3. The method of embodiment 1 or 2, wherein B does not bind the capture reagent.
4. The method of any one of embodiments 1-3, wherein the plurality of capture reagents is one or more of an antigen binding protein, an antibody, a soluble receptor, a protein A, a protein G, a protein L, or an extracellular domain.
5. The method of any one of embodiments 1-4, wherein one or more of the capture agents is immobilized on a solid support.
6. The method of embodiment 5, wherein the solid support is a bead.
7. The method of any one of embodiments 1-6, wherein the base detection moiety is an antigen binding protein, an antibody, or a soluble receptor.
8. The method of any one of embodiments 1-7, wherein n is 6-18.
9. The method of any one of embodiments 1-8, wherein p is 1-8.
10. The method of any one of embodiment 1-9, wherein the linker is cleavable by an enzyme or by a chemical.
11. The method of any one of embodiments 1-10, wherein the base detection moiety is not cleaved under the conditions to cleave the linker.
12. The method of embodiment 10 or 11, wherein the enzyme is a protease.
13. The method of embodiment 12, wherein the protease is an endopeptidase.
14. The method of embodiment 13, wherein the endopeptidase is papain.
15. The method of any one of embodiments 10-14, wherein the base detection moiety does not comprise an amino acid sequence that undergoes chemical cleavage or protease cleavage.
16. The method of any of embodiments 1-15, wherein the tag is one or more of:
   a) a molecule that can be ionized and vaporized for analysis by mass spectrometry,
   b) a molecule with a controlled conjugation site,
   c) unique from other peptides in the sample,
   d) provides a good LC-MS response,
   e) does not compromise binding to the analyte when bound to base,
   f) has the capacity to functionalize for a multiplexed assay; and
   g) monitorable by a precursor ion-product ion pair ranging from 0-2000 m/z→0-2000 m/z.
17. The method of any one of embodiments 1-16, wherein the detection reagent binds the analyte at a different site than the capture polypeptide.
18. A method for detecting an analyte in a sample, the method comprising
   a) contacting the sample with a capture reagent to generate a captured analyte complex, wherein the capture reagent comprises a binding reagent that binds the analyte, and wherein the capture antibody is immobilized on a solid support,
   b) contacting the captured analyte complex with a detection reagent, wherein the detection reagent binds the captured analyte complex, wherein the detection reagent is a compound having the formula

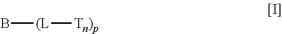

wherein:
   B is a base detection moiety that binds the analyte,
   L is a cleavable linker,
   T is a tag suitable for mass spectroscopy,
   n is an integer greater than or equal to 1, and
   p is an integer greater than or equal to 1;
   c) washing the captured analyte complex with a buffer to remove unbound detection reagent;
   d) subjecting the sample from step c) to papain to cleave the linker to release the tag from the detection antibody,
   e) analyzing the sample from step d) for presence of the released tag by mass spectroscopy.
19. A method for quantifying an analyte in a sample, the method comprising
   a) contacting the sample with a capture reagent to generate a captured analyte complex, wherein the capture reagent comprises a binding reagent that binds the analyte, and wherein the capture antibody is immobilized on a solid support,
   b) contacting the captured analyte complex with a detection reagent, wherein the detection reagent binds the captured analyte complex, wherein the detection reagent is a compound having the formula

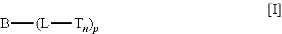

wherein:
   B is a base detection moiety that binds the analyte,
   L is a cleavable linker,
   T is a tag suitable for mass spectroscopy,
      n is an integer greater than or equal to 1, and
      p is an integer greater than or equal to 1;
   c) washing the captured analyte complex with a buffer to remove unbound detection reagent;
   d) subjecting the sample from step c) to papain to cleave the linker to release the tag from the detection antibody,
   e) quantifying the released tag by mass spectroscopy.
20. The method of any one of embodiments 1-19, wherein the analyte is a therapeutic polypeptide, a therapeutic antibody, or a biomarker.
21. A method for detecting a plurality of analytes in a sample, the method comprising
   a) contacting the sample with a plurality of capture reagents to generate a plurality of captured analyte complexes, wherein the plurality of capture reagents comprises a plurality of capture polypeptides that bind the plurality of analytes,
   b) contacting the plurality of captured analyte complexes with a plurality of detection reagents, wherein the plurality of detection reagents bind the plurality of captured analyte complexes, wherein each detection reagent in the plurality of detection reagents is a compound having the formula

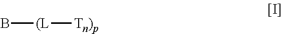

wherein:
   B is a base detection moiety that binds the analyte,
   L is a cleavable linker,
   T is a tag suitable for mass spectroscopy,
   n is an integer greater than or equal to 1, and
   p is an integer greater than or equal to 1,
   wherein each tag in the plurality of detection reagents can be distinguished from one another by mass spectroscopy;
   c) washing the plurality of captured analyte complex with a buffer to remove unbound detection reagent;
   d) subjecting the sample from step c) under conditions to cleave the linker to release the tags from the plurality of detection reagents,
   e) analyzing the sample from step d) for presence of the plurality of released tags by mass spectroscopy.
22. A method for quantifying a plurality of analytes in a sample, the method comprising
   a) contacting the sample with a plurality of capture reagents to generate a plurality of captured analyte complexes, wherein the plurality of capture reagents comprises a plurality of capture polypeptides that bind the plurality of analytes,
   b) contacting the plurality of captured analyte complexes with a plurality of detection reagents, wherein the plurality of detection reagents bind the plurality of captured analyte complexes, wherein each detection reagent in the plurality of detection reagents is a compound having the formula

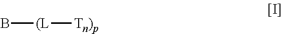

wherein:
   B is a base detection moiety that binds the analyte,
   L is a cleavable linker,
   T is a tag suitable for mass spectroscopy,
   n is an integer greater than or equal to 1, and
   p is an integer greater than or equal to 1,
   wherein each tag in the plurality of detection reagents can be distinguished from one another by mass spectroscopy;
   c) washing the captured analyte complex with a buffer to remove unbound detection reagent;
   d) subjecting the sample from step c) under conditions to cleave the linker to release the tags from the plurality of detection reagents,
   e) quantifying the plurality of released tags by mass spectroscopy.
23. The method of embodiment 21 or 22, wherein B does not bind the capture reagent.
24. The method of any one of embodiments 21-23, wherein the plurality of capture reagents is one or more of an antigen binding protein, an antibody, a soluble receptor, a protein A, a protein G, a protein L, or an extracellular domain.
25. The method of any one of embodiments 21-24, wherein one or more of the capture agents is immobilized on a solid support.
26. The method of embodiment 25, wherein the solid support is a bead.
27. The method of any one of embodiments 21-26, wherein the base detection moiety is an antigen binding protein, an antibody, or a soluble receptor.
28. The method of any one of embodiments 21-27, wherein n is 6-18.
29. The method of any one of embodiments 21-28, wherein p is 1-8.
30. The method of any one of embodiment 21-29, wherein the linkers of the plurality of detection reagents are cleavable by enzymes or by chemicals.
31. The method of any one of embodiments 21-30, wherein the plurality of base detection moieties are not cleaved under the conditions to cleave the linker.
32. The method of embodiment 30 or 31, wherein the enzyme is a protease.
33. The method of any one of embodiments 21-32, wherein linkers of the plurality of detection reagents are cleavable by one or more proteases.
34. The method of embodiment 33, wherein one or more of the proteases is an endopeptidase.
35. The method of embodiment 33, wherein the one or more endopeptidases is papain.
36. The method of any one of embodiments 30-35, wherein one or more of the base detection moieties of the plurality of base detection moieties do not comprise an amino acid sequence that undergoes chemical cleavage or protease cleavage.
37. The method of any one of embodiments 21-36, wherein one or more of the linkers in the plurality of detection reagents are cleaved by the same protease or chemical.
38. The method of any one of embodiments 21-37, wherein all the linkers in the plurality of detection reagents are the same.
39. The method of any of embodiments 21-38, wherein the tag for each detection reagent of the plurality of detection reagents is one or more of
   a) a molecule that can be ionized and vaporized for analysis by mass spectrometry,
   b) a molecule with a controlled conjugation site,
   c) unique from other peptides in the sample,
   d) provides a good LC-MS response,
   e) does not compromise binding to the analyte when bound to base, or
   f) has the capacity to functionalize for a multiplexed assay.
40. A method for detecting a plurality of analytes in a sample, the method comprising
   a) contacting the sample with a plurality of capture reagents to generate a plurality of captured analyte complexes, wherein the plurality of capture reagents comprises a plurality of capture antibodies that bind the plurality of analytes, and wherein the plurality of capture antibodies are immobilized on a solid support,
   b) contacting the plurality of captured analyte complexes with a plurality of detection reagents, wherein the plurality of detection reagents bind the plurality of captured analyte complexes, wherein each detection reagent in the plurality of detection reagents is a compound having the formula

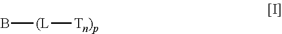

wherein:
   B is a base detection moiety that binds the analyte,
   L is a cleavable linker,
   T is a tag suitable for mass spectroscopy,
   n is an integer between 1 and 50, and
   p is an integer between 1 and 50,
   wherein each tag in the plurality of detection reagents can be distinguished from one another by mass spectroscopy;
   c) washing the plurality of captured analyte complexes with a buffer to remove unbound detection reagents;
   d) subjecting the sample from step c) to papain to cleave the linker to release the tags from the plurality of detection reagents, and
   e) analyzing the sample from step d) for presence of the plurality of released tags by mass spectroscopy.
41. A method for quantifying a plurality of analytes in a sample, the method comprising
   a) contacting the sample with a plurality of capture reagents to generate a plurality of captured analyte complexes, wherein the plurality of capture reagents comprises a plurality of capture antibodies that bind the plurality of analytes, and wherein the plurality of capture antibodies are immobilized on a solid support,
   b) contacting the plurality of captured analyte complexes with a plurality of detection reagents, wherein the plurality of detection reagents bind the plurality of captured analyte complexes, wherein each detection reagent in the plurality of detection reagents is a compound having the formula

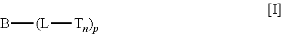

wherein:
   B is a base detection moiety that binds the analyte,
   L is a cleavable linker,
   T is a tag suitable for mass spectroscopy,
   n is an integer between 1 and 6, and
   p is an integer between 1 and 8,
   wherein each tag in the plurality of detection reagents can be distinguished from one another by mass spectroscopy;
   c) washing the plurality of captured analyte complexes with a buffer to remove unbound detection reagents;
   d) subjecting the sample from step c) to papain to cleave the linker to release the tags from the plurality of detection reagents, and
   e) quantifying the plurality of released tags by mass spectroscopy.
42. The method of any one of embodiments 21-41, wherein the plurality of analytes comprise two or more of therapeutic polypeptides, therapeutic antibodies, and/or biomarkers.
43. A detection reagent for use in detecting an analyte in a sample by mass spectroscopy, wherein the detection reagent is a compound having the formula

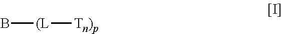

wherein:
   B is a base detection moiety that binds the analyte,
   L is a cleavable linker,
   T is a tag suitable for mass spectroscopy,
   n is an integer greater than or equal to 1, and
   p is an integer greater than or equal to 1.
44. The detection reagent of embodiment 43, wherein the base detection moiety is an antigen binding protein, an antibody, or a soluble receptor.
45. The detection reagent of embodiment 43 or 44, wherein n is 6-18.
46. The detection reagent of any one of embodiments 43-45, wherein p is 1-8.
47. The detection reagent of any one of embodiments 43-46, wherein the linker is cleavable by an enzyme or by a chemical.
48. The detection reagent of any one of embodiments 43-47, wherein the base detection moiety is not cleaved under the conditions to cleave the linker.
49. The detection reagent of embodiment 47 or 48, wherein the enzyme is a protease.
50. The detection reagent of embodiment 49, wherein the protease is an endopeptidase.
51. The detection reagent of embodiment 50, wherein the endopeptidase is papain.
52. The detection reagent of any one of embodiments 43-51, wherein the base detection moiety does not comprise an amino acid sequence that undergoes chemical cleavage or protease cleavage.
53. The detection reagent of any one of embodiments 43-52, wherein the tag is one or more of
   a) a molecule that can be ionized and vaporized for analysis by mass spectrometry,
   b) a molecule with a controlled conjugation site,
   c) unique from other peptides in the sample,
   d) provides a good LC-MS response,
   e) does not compromise binding to the analyte when bound to base, or
   f) has the capacity to functionalize for a multiplexed assay.
54. The detection reagent of any one of embodiments 43-53, wherein the detection reagent binds the analyte at a different site than the capture polypeptide.
55. A detection reagent for use in detecting an analyte in a sample by mass spectroscopy,
   wherein the detection reagent is a compound having the formula

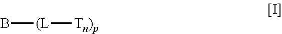

wherein:
   B is a base detection moiety that binds the analyte,
   L is a cleavable linker,
   T is a tag suitable for mass spectroscopy,
   n is an integer greater than or equal to 1, and
      p is an integer greater than or equal to 1.
56. The detection reagent of any one of embodiments 43-55, wherein the plurality of analytes are two or more of therapeutic polypeptides, therapeutic antibodies, and/or biomarkers.
57. A composition comprising the detection reagent of any one of embodiments 43-56.
58. A composition comprising a plurality of detection reagents of any one of embodiments 43-56, wherein each detection reagent in the plurality of detection reagents binds a different analyte and comprises a different tag.
59. A kit for use in the method of any one of embodiments 1-42.
60. A kit comprising the detection reagent of any one of embodiments 43-56.
61. A kit comprising the composition of embodiment 57 or 58.
62. A method for detecting an analyte in a sample, comprising:
   a) contacting the sample with a capture reagent to generate a captured analyte complex, wherein the capture reagent comprises a binding reagent that binds the analyte,
   b) contacting the captured analyte complex with a detection reagent, wherein the detection reagent binds the captured analyte complex, wherein the detection reagent comprises a base detection moiety that binds the analyte, wherein the base detection moiety is directly or indirectly linked to a plurality of tags, and wherein the plurality of tags are cleavable from the detection reagent;
   c) separating unbound detection reagent from the captured analyte complex bound to the detection reagent;
   d) cleaving the plurality of tags from the detection reagent bound to the captured analyte complex, thereby releasing the tags, and
   e) analyzing the released tags by mass spectroscopy.
63. The method of embodiment 62, wherein analyzing the released tags by mass spectroscopy comprises generating a signal-to-noise ratio (S/N) of the released tags.
64. The method of embodiment 63, wherein the S/N of the released tags detected by mass spectroscopy is about 2, 3, 4, 5, 6, 7, 8, 9, 10, 11, 12, 13, 14, 15, 16, 17, 18, 19, 20, or more.
65. The method of any of embodiments 62-64, wherein the lower limit of quantitation (LLOQ) for the analyte is between about 0.1 and about 20 ng/mL, between about 0.2 and about 15 ng/ml, or between about 0.5 and about 10 ng/mL.
66. The detection reagent or method of any one of embodiments 1-65, wherein the linker comprises a branched linker.
67. The detection reagent or method of embodiment 66, wherein the branched linker comprises one or more primary linkers and one or more secondary linkers.
68. The detection reagent or method of embodiment 67, wherein the one or more primary linkers comprise one or more branching points.
69. The detection reagent or method of embodiment 67 or 68, wherein the one or more secondary linkers are fused to the one or more primary linkers at the one or more branching points.
70. The detection reagent or method of embodiment 66, wherein the branched linker comprises one or more primary linkers, one or more secondary linkers, and one or more tertiary linkers.
71. The detection reagent or method of embodiment 70, wherein the one or more primary linkers comprise one or more branching points.
72. The detection reagent or method of embodiment 71, wherein the one or more secondary linkers comprise one or more branching points.
73. The detection reagent or method of any one of embodiments 70-72, wherein the one or more tertiary linkers are fused to the one or more secondary linkers at the one or more branching points.
74. The detection reagent or method of any one of embodiments 70-73, wherein the one or more secondary linkers are fused to the one or more primary linkers at the one or more branching points.
75. The detection reagent or method of any one of embodiments 1-65, wherein the tag comprises a polypeptide.
76. The detection reagent or method of embodiment 75, wherein the polypeptide comprises a contiguous amino acid sequence.
77. The detection reagent or method of embodiment 76, wherein the amino acid sequence comprises a length between about 2 to 100 amino acids.
78. The detection reagent or method of embodiment 76 or 77, wherein the amino acid sequence comprises a length between about 5 to 10 amino acids.
79. The detection reagent or method of any one of embodiments 75-78, wherein the polypeptide comprises leucine enkephalin.
80. The detection reagent or method of any one of embodiments 75-78, wherein the polypeptide comprises valine-citrulline.
81. The detection reagent or method of any one of embodiments 1-65, wherein the tag comprises a small molecule.
82. The detection reagent or method of embodiment 81, wherein the small molecule is selected from the group consisting of an auristatin, maytansinoid, calicheamicin, and camptothecin.
83. The detection reagent or method of embodiment 82, wherein the auristatin comprises monomethyl auristatin E (MMAE).
84. The detection reagent or method of any one of embodiments 1-65, wherein the tag comprises an oligonucleotide.
85. The detection reagent or method of embodiment 84, wherein the oligonucleotide comprises RNA.
86. The detection reagent or method of embodiment 84, wherein the oligonucleotide comprises DNA.
87. The detection reagent or method of any one of embodiments 84-86, wherein the oligonucleotide comprises a contiguous nucleotide sequence.
88. The detection reagent or method of embodiment 87, wherein the oligonucleotide comprises a length between about 2 to 100 nucleotides.

### EXAMPLES

The present disclosure will be more fully understood by reference to the following examples. They should not, however, be construed as limiting the scope of the present disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: Quantification of a Polypeptide Analyte Using Detector Reagents with Variable Number of Tag Molecules

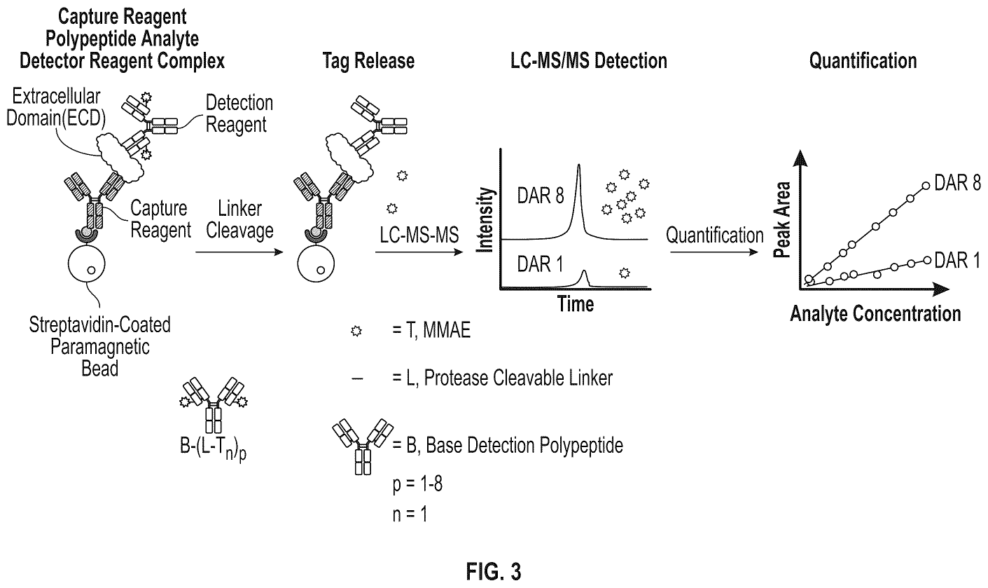

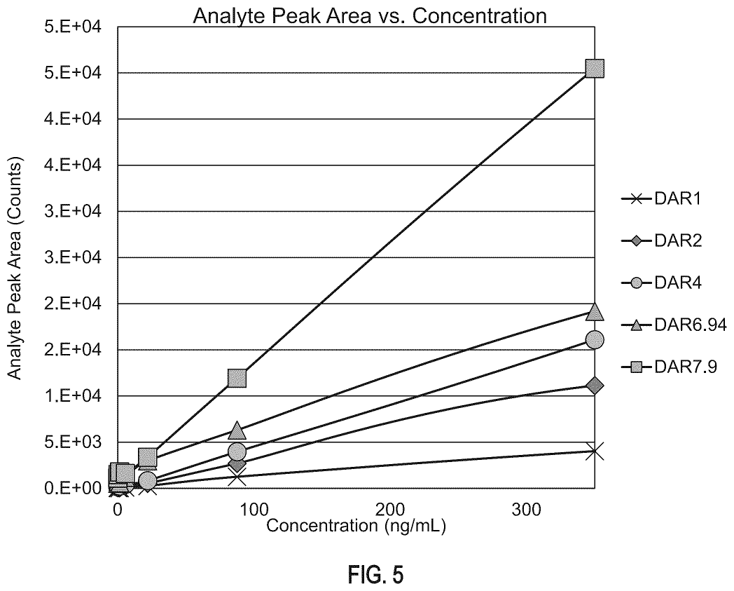

In the example below, the following components are used: polypeptide analyte: a receptor extracellular domain (ECD); base detection moiety: an antibody against the receptor; tag: MMAE; capture reagent: a biotinylated antibody against the receptor. The receptor ECD was provided in a buffer comprising phosphate buffered saline (PBS) with 0.5% BSA, 0.05% Proclin300, 0.05% P20, 0.25% CHAPS in 5 mM EDTA and 0.35M NaCl, pH 7.4. The buffer was analyzed by the following immunoaffinity bead capture and mass spectrometry methods to measure the relative amounts of the polypeptide analyte using surrogate tag molecules. FIG. 3 shows a schematic representation of capture of the polypeptide analyte on streptavidin coated magnetic beads bound to a biotinylated capture antibody, followed by isolation of the polypeptide analyte by magnetic separation, addition of a detection reagent conjugated to a variable number of tags (MMAE), cleavage of the tags, and analysis by LC-MS/MS using MMAE as the surrogate tag for quantification of the polypeptide analyte. This method successfully identified the polypeptide analyte sample constituents in the concentration range tested (0.25-1400 ng/ml) in a sample volume ranging from 10 to 100 μL. Detection reagents with a different number of tags bound to the detecting polypeptide were used as shown in Table 1. FIG. 5 shows the linearity of detection, by plotting the peak area counts of tag (MMAE) versus the concentration of the polypeptide analyte. Increasing the numbers of tag molecules in the detector reagents results in higher analyte peak area counts.

TABLE 1
Detection reagents comprising increasing Tag numbers.

| Name | Drug-to-Antibody Ratio (DAR) |
|---|---|
| Detection reagent 1 | 1.00 |
| Detection reagent 2 | 2.00 |
| Detection reagent 3 | 4.00 |
| Detection reagent 5 | 6.94 |
| Detection reagent 6 | 7.9 |

Exemplary detection reagents include those disclosed in U.S. Pat. No. 8,541,178, incorporated herein by reference in its entirety for all purposes.

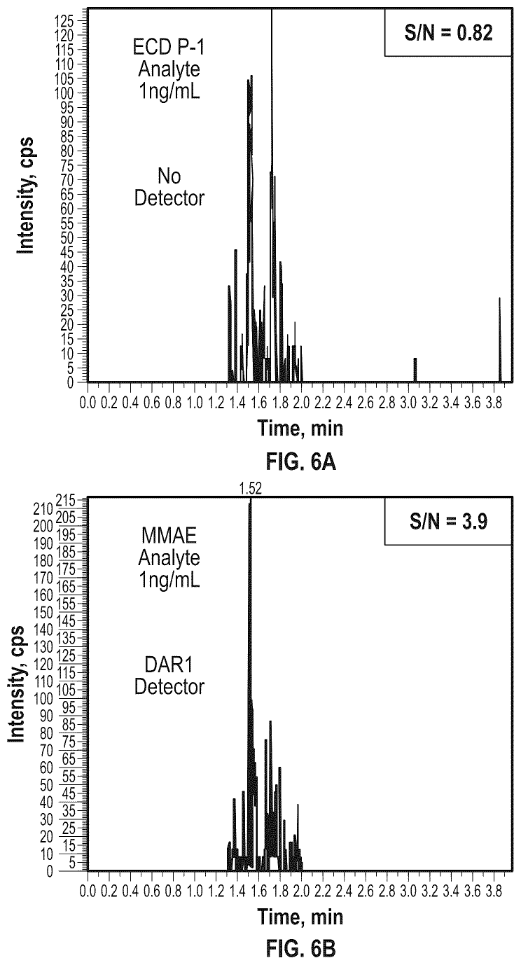

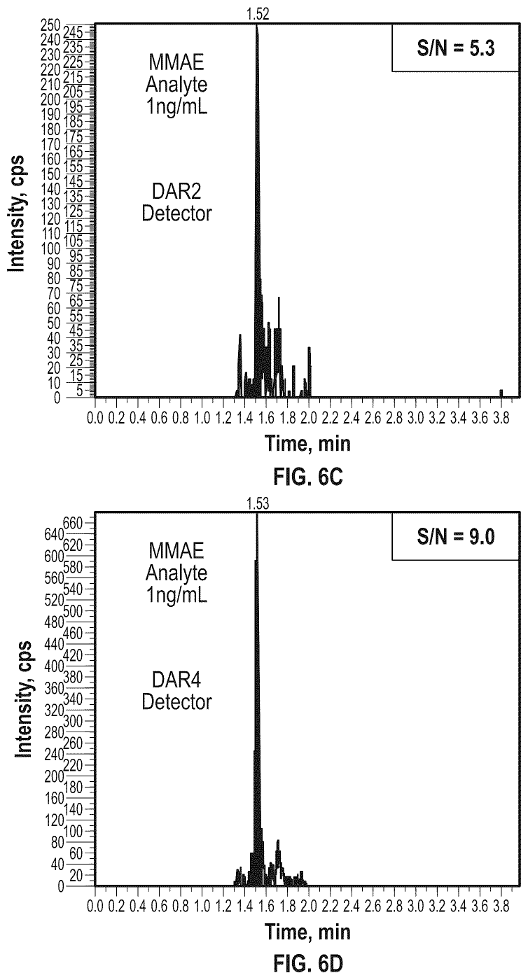

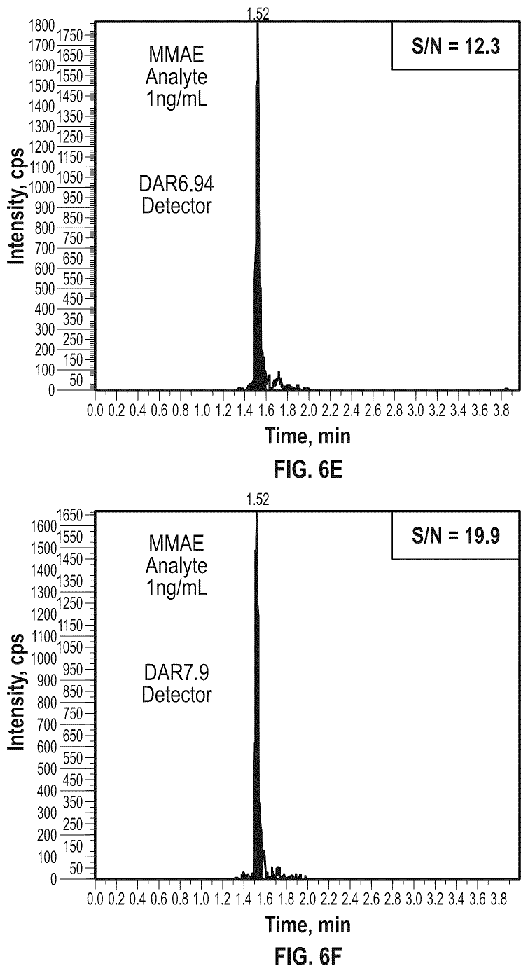

Comparison of the detection reagents and surrogate tryptic peptide method were made by employing a set of samples and capture reagents at the same concentrations. Standard curves of the polypeptide analyte using quantification of the analyte directly with a surrogate peptide and using quantification with additional detector reagent were generated. The limit of quantitation was 40 times lower for the quantification using additional detection reagents. FIGS. 6A-6F shows a comparison of the extracted ion chromatograms of MMAE (tag from detector reagent) and ECD-P1 surrogate peptide from the polypeptide analyte at the LLOQ level for traditional method (1 ng/mL). Use of MMAE increased the signal/noise ratio compared to use of the surrogate peptide. Thus, use of a homogeneous population of tag molecules (e.g., MMAE) in the exemplary methods provided herein achieved greater sensitivity compared to use of a heterogeneous population of peptide molecules (e.g., peptides from ECD P1 analyte), and increasing the number of tag molecules in the detection reagents can be used to achieve greater sensitivity. Typically a signal-to-noise ratio (S/N) in detection is greater than about 3, greater than about 4, or greater than about 5 to allow the peak to be considered above the limit of detection, as an indicator of signal differentiating against the background.

### Example 2: Protocol for Quantification of a Polypeptide Analyte Using Detector Reagents with Variable Number of Tag Molecules

PBS with 0.5% BSA, 0.05% Proclin300, 0.05% P20, 0.25% CHAPS in 5 mM EDTA and 0.35M NaCl, pH 7.4 spiked with the polypeptide analyte (a receptor extracellular domain (ECD)) was processed by the following steps:

The polypeptide analyte was fortified into phosphate buffered saline (PBS) with 0.5% BSA, 0.05% Proclin300, 0.05% P20, 0.25% CHAPS in 5 mM EDTA and 0.35M NaCl, pH 7.4 solution for testing. Immunoaffinity enrichment was conducted with streptavidin-coated Dynabeads& M-280 (ThermoFisher, Waltham, MA, USA). Bead suspension (25 μL) was added to each well of a 96-well plate. Using an automated washing system Kingfisher Flex™ (ThermoFisher, Waltham, MA, USA), the beads were washed two-times with 300 μL of phosphate buffered saline with 0.5% BSA, 0.05% Proclin300, 0.05% P20, 0.25% CHAPS in 5 mM EDTA and 0.35M NaCl, pH 7.4, and then resuspended with 200 μL of phosphate buffered saline with 0.5% BSA, 0.05% Proclin300, 0.05% P20, 0.25% CHAPS in 5 mM EDTA and 0.35M NaCl, pH 7.4 containing 2 μg of biotinylated antibody against the receptor. The plate was incubated at RT for 60 minutes in a Microplate Shaker (Eppendorf, Hamburg, Germany) at a vortexing speed of between 8-9 setting. The beads were then washed three-times with 500 μL of phosphate buffered saline with 0.5% BSA, 0.05% Proclin300, 0.05% P20,0.25% CHAPS in 5 mM EDTA and 0.35M NaCl, pH 7.4, and then resuspended with 200 μL of phosphate buffered saline with 0.5% BSA, 0.05% Proclin300, 0.05% P20, 0.25% CHAPS in 5 mM EDTA and 0.35M NaCl, pH 7.4 and 100 μL of the sample, QC, standards, or blanks. The plate was incubated at RT for 60 minutes in a Microplate Shaker at a vortexing speed of between 8-9 setting. The beads were then washed three-times with 500 μL of phosphate buffered saline with 0.5% BSA, 0.05% Proclin300, 0.05% P20, 0.25% CHAPS in 5 mM EDTA and 0.35M NaCl, pH 7.4, and then resuspended into 250 μL of phosphate buffered saline with 0.5% BSA, 0.05% Proclin300, 0.05% P20, 0.25% CHAPS in 5 mM EDTA and 0.35M NaCl, pH 7.4 containing 50 μL of 200 mM detector (an antibody conjugated to tags). The plate was incubated at RT for 60 minutes in a Microplate Shaker at a vortexing speed of 1200 rpm. The beads were then washed two-times with 200 μL of HBS-EP buffer with 0.1% BSA. Following incubation, the plate was washed two times with 500 μL of PBS to remove any non-specifically bound proteins. The cleavage of the linker was initiated by adding 120 μL of papain in papain activating buffer (2.5 mg/ml in 2 mM L-Cysteine in 50 mM ammonium acetate, pH 7.0). The mixture was incubated at 25° C. for 60 minutes. The plate was centrifuged for 10 minutes at 4000 rpm to collect the 100 μL of the supernatant. The supernatant was evaporated to dryness with nitrogen at 45° C. The samples were resuspended in 20 μL 80/20 H₂O/MeCN followed by 80 μL MeOH containing 0.5 nM d-8 MMAE for protein precipitation. The supernatant was then analyzed using LC-MS/MS.

LC-MS/MS was carried out using a Shimadzu (Tokyo, Japan) Nexera HPLC system interfaced to a SCIEX QTRAP® 5500 (AB Sciex, Concord, ON). For the analysis of released tag analyte, Phenomenex Synergi RP-Max (4 μm, 2×50 mm, Torrance, CA, USA) was used for chromatographic separation and the mobile phases consisted of mobile phase A (5 mM ammonium acetate+0.1% formic acid) and mobile phase B (5 mM ammonium acetate in 5:95 water:ACN with 0.1% formic acid). The LC gradient was 0%-100% B in 2.8 min, 100% B in 0.2 min, 0% B in 0.2 min, 0%-100% B in 0.2 min, 100% B in 0.2 min 100%-0% B in 0.1 min and 0% B in 0.3 min with a flow rate of 0.5 mLmin-1. The volume of 10 μl was loaded on the column whose temperature was maintained at 50° C. Positive ion electrospray mass spectrometry in multiple reaction monitoring (MRM) mode was used for the detection of analyte. The source temperature and turbo ionspray voltage were set to 550° C. and 5000 V, respectively. For the analysis of released drug analyte, the transitions of m/z 718.433→152.099 and m/z 726.570→152.107 were monitored for MMAE and the MMAE-d8 respectively.

### Example 3: Quantification of a Polypeptide Analyte Using a Detector Reagent with Leucine Enkephalin Tags

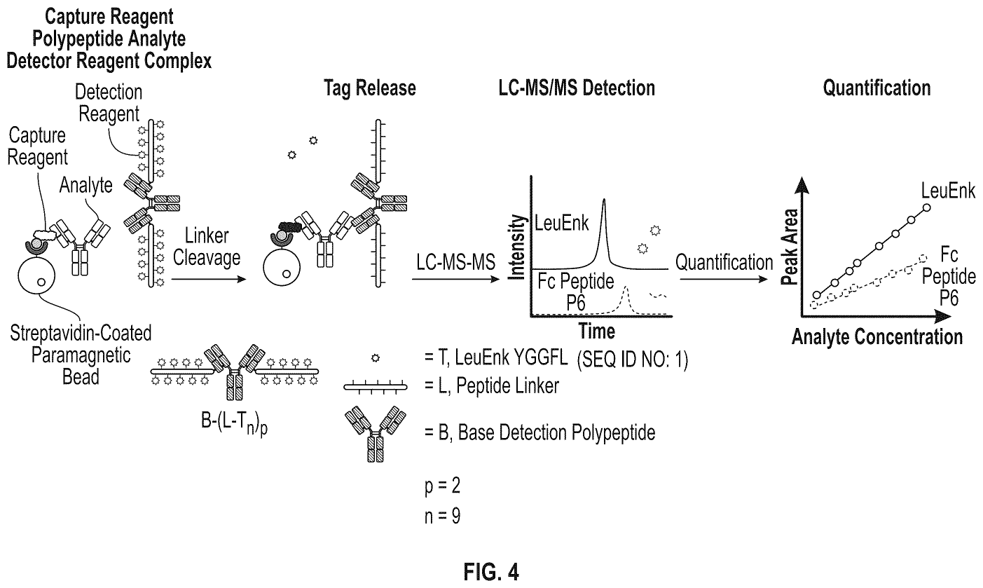

In this example, the following components are used; a polypeptide analyte: an antibody against a receptor; a base detection moiety: a humanized antibody conjugated to 18 leucine enkephalins; tag: leucine enkephalin; capture reagent: biotinylated receptor extracellular domain (ECD). The polypeptide analyte is provided in HBS-EP buffer (0.01 M HEPES pH 7.4, 0.15 M NaCl, 3 mM EDTA, 0.005% v/v polysorbate 20) with 0.1% BSA and is analyzed by immunoaffinity bead capture and mass spectrometry methods to measure the relative amounts of the polypeptide analyte using surrogate tag molecules (leucine enkephalin; SEQ ID NO:1). FIG. 4 shows a schematic representation of capture of the polypeptide analyte on streptavidin coated magnetic beads bound to a biotinylated capture reagent, followed by isolation of the polypeptide analyte by magnetic separation, addition of a detector reagent with 18 leucine enkephalins, cleavage of the leucine enkephalins and analysis by LC-MS/MS using leucine enkephalin as the surrogate tag for quantification of the polypeptide analyte. This method successfully identifies the polypeptide analyte sample constituents in the 9.75-10000 ng/mL concentration range in a sample volume of 100 μL.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1. A method for detecting an analyte (A) in a sample, the method comprising
a) affinity enrichment by contacting the sample with a capture reagent to generate a captured analyte complex, wherein the capture reagent comprises a binding reagent that binds the analyte,
b) contacting the captured analyte complex with a detection reagent, wherein the detection reagent binds the captured analyte complex, wherein the detection reagent is a compound having the formula 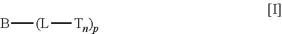
wherein:
B is a base detection moiety that binds the analyte,
L is a cleavable linker,
T is a tag suitable for mass spectroscopy,
n is an integer greater than or equal to 1, and
p is an integer greater than or equal to 1;
c) washing the captured analyte complex with a buffer to remove unbound detection reagent;
d) subjecting the sample from step c) under conditions to cleave the linker to release the tag, and
e) analyzing the sample from step d) for presence of the released tag by mass spectroscopy.

2. (canceled)

3. The method of claim 1, wherein B does not bind the capture reagent.

4. The method of claim 1, wherein the plurality of capture reagents is one or more of an antigen binding protein, an antibody, a soluble receptor, a protein A, a protein G, a protein L, or an extracellular domain.

5. The method of claim 1, wherein one or more of the capture agents is immobilized on a solid support.

6. The method of claim 5, wherein the solid support is a bead.

7. The method of claim 1, wherein the base detection moiety is an antigen binding protein, an antibody, or a soluble receptor.

8. The method of claim 1, wherein n is 6-18.

9. The method of claim 1, wherein p is 1-8.

10. The method of claim 1, wherein the linker is cleavable by an enzyme or by a chemical.

11. The method of claim 1, wherein the base detection moiety is not cleaved under the conditions to cleave the linker.

12. The method of claim 10, wherein the enzyme is a protease.

13. The method of claim 12, wherein the protease is an endopeptidase.

14. The method of claim 13, wherein the endopeptidase is papain.

15. The method of claim 10, wherein the base detection moiety does not comprise an amino acid sequence that undergoes chemical cleavage or protease cleavage.

16. The method of claim 1, wherein the tag is one or more of:
a) a molecule that can be ionized and vaporized for analysis by mass spectrometry,
b) a molecule with a controlled conjugation site,
c) unique from other peptides in the sample,
d) provides a good LC-MS response,
e) does not compromise binding to the analyte when bound to base,
f) has the capacity to functionalize for a multiplexed assay; and
g) monitorable by a precursor ion-product ion pair ranging from 0-2000 m/z→0-2000 m/z.

17. The method of claim 1, wherein the detection reagent binds the analyte at a different site than the capture polypeptide.

18.-19. (canceled)

20. The method of claim 1, wherein the analyte is a therapeutic polypeptide, a therapeutic antibody, or a biomarker.

21. A method for detecting a plurality of analytes in a sample, the method comprising
a) contacting the sample with a plurality of capture reagents to generate a plurality of captured analyte complexes, wherein the plurality of capture reagents comprises a plurality of capture polypeptides that bind the plurality of analytes,
b) contacting the plurality of captured analyte complexes with a plurality of detection reagents, wherein the plurality of detection reagents bind the plurality of captured analyte complexes, wherein each detection reagent in the plurality of detection reagents is a compound having the formula 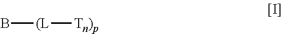
wherein:
B is a base detection moiety that binds the analyte,
L is a cleavable linker,
T is a tag suitable for mass spectroscopy,
n is an integer greater than or equal to 1, and
p is an integer greater than or equal to 1,
wherein each tag in the plurality of detection reagents can be distinguished from one another by mass spectroscopy;
c) washing the plurality of captured analyte complex with a buffer to remove unbound detection reagent;
d) subjecting the sample from step c) under conditions to cleave the linker to release the tags from the plurality of detection reagents,
e) analyzing the sample from step d) for presence of the plurality of released tags by mass spectroscopy.

22.-61. (canceled)

62. A method for detecting an analyte in a sample, comprising:
a) contacting the sample with a capture reagent to generate a captured analyte complex, wherein the capture reagent comprises a binding reagent that binds the analyte,
b) contacting the captured analyte complex with a detection reagent, wherein the detection reagent binds the captured analyte complex, wherein the detection reagent comprises a base detection moiety that binds the analyte, wherein the base detection moiety is directly or indirectly linked to a plurality of tags, and wherein the plurality of tags are cleavable from the detection reagent;
c) separating unbound detection reagent from the captured analyte complex bound to the detection reagent;
d) cleaving the plurality of tags from the detection reagent bound to the captured analyte complex, thereby releasing the tags, and
e) analyzing the released tags by mass spectroscopy.

63.-65. (canceled)

66. The method of claim 1, further comprising quantifying the analyte (A) by quantifying the released tag by mass spectroscopy.


SEQUENCE LISTING (1 sequence)

SEQ ID NO: 1 - protein, 5 aa, synthetic construct
  REGION 1..5: note = Leucine Enkephalin tag
YGGFL